(19) United States
(12) Patent Application Publication
(10) Pub. No.: US 2008/0171755 A1
(43) Pub. Date: Jul. 17, 2008

(54) Pyrimidinylpyrazoles as Tgf-Beta Inhibitors

(76) Inventors: Wen-Cherng Lee, Lexington, MA (US); Lihong Sun, Arlington, MA (US); Claudio Chuaqui, Somerville, MA (US); Mark Comebise, Watertown, MA (US); Juswinder Singh, Ashand, MA (US); Feng Shan, Braintree, MA (US)

Correspondence Address: MILLER, CANFIELD, PADDOCK AND STONE, PLC, 277 SOUTH ROSE STREET, SUITE 5000, KALAMAZOO, MI, 49007, US

(21) Appl. No.: 11/661,531

(22) PCT Filed: Aug. 24, 2005
(86) PCT No.: PCT/US2005/030132

§ 371 Date: Feb. 28, 2008

Related U.S. Application Data
(60) Provisional application No. 60/606,046, filed on Aug. 31, 2004.

(51) Int. Cl. A61K 31/506; C07D 405/14; C12N 5/06; A61P 35/00; A61P 35/02; A61P 25/28

(52) U.S. Cl. 514/256; 544/333; 435/375

(57) ABSTRACT

The invention is based on the discovery that compounds of formula (I) possess high affinity for Alk 5 and/or Alk 4, and can be useful as antagonists thereof for preventing and/or treating numerous diseases, including fibrotic disorders. The invention features a compound of formula (I) and uses thereof: formula (I).

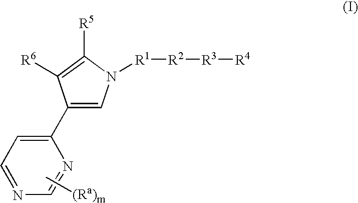

This application claims priority to U.S. Ser. No. 60/606,046 which was filed on Aug. 31, 2004. The entire content of the aforementioned application is incorporated in its entirety.

### BACKGROUND OF THE INVENTION

TGFβ (Transforming Growth Factor β) is a member of a large family of dimeric polypeptide growth factors that includes activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3) and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptors and is not biologically active. TGFβ is generally released (and activated) from the complex by a variety of mechanisms including, for example, interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk 5) in a glycine/serine rich domain, which allows the type I receptor to recruit and phosphorylate downstream signaling molecules, Smad2 or Smad3. See, e.g., Huse, M. et al., Mol. Cell. 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J. Ann. Rev. Biochem. Med. 67: 773 (1998).

Activins are also members of the TGFβ superfamily which are distinct from TGFβ in that they are homo- or heterodimers of activin βa or βb. Activins signal in a similar manner to TGFβ, that is, by binding to a constitutive serine-threonine receptor kinase, activin type II receptor (ActRIIB), and activating a type I serine-threonine receptor, Alk 4, to phosphorylate Smad2 or Smad3. The consequent formation of a hetero-Smad complex with Smad4 also results in the activin-induced regulation of gene transcription.

Indeed, TGFβ and related factors such as activin regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J. Ann. Rev. Cell. Biol. 6: 594-641 (1990); Roberts, A. B. and Sporn M. B. Peptide Growth Factors and Their Receptors, 95: 419-472 Berlin: Springer-Verlag (1990); Roberts, A. B. and Sporn M. B. Growth Factors 8:1-9 (1993); and Alexandrow, M. G., Moses, H. L. Cancer Res. 55: 1452-1457 (1995). Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and overexpression of activin is linked to pathological disorders that involve extracellular matrix accumulation and fibrosis (see, e.g., Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); Sugiyama, M. et al., Gastroenterology 114: 550-558 (1998); Munz, B. et al., EMBO J. 18: 5205-5215 (1999)), inflammatory responses (see, e.g., Rosendahl, A. et al., Am. J. Repir. Cell Mol. Biol. 25: 60-68 (2001)), cachexia or wasting (see Matzuk, M. M. et al., Proc. Nat. Acad. Sci. USA 91: 8817-8821 (1994); Coerver, K. A. et al., Mol. Endocrinol. 10: 534-543 (1996); Cipriano, S. C. et al. Endocrinology 141: 2319-27 (2000)), diseases of or pathological responses in the central nervous system (see Logan, A. et al. Eur. J. Neurosci. 11: 2367-2374 (1999); Logan, A. et al. Exp. Neurol. 159: 504-510 (1999); Masliah, E. et al., Neurochem. Int. 39: 393-400 (2001); De Groot, C. J. A. et al, J. Neuropathol. Exp. Neurol. 58: 174-187 (1999), John, G. R. et al, Nat. Med. 8: 1115-21 (2002)) and hypertension (see Dahly, A. J. et al., Am. J. Physiol. Regul. Integr. Comp. Physiol. 283: R757-67 (2002)). Studies have also shown that TGFβ and activin can act synergistically to induce extracellular matrix (see, e.g., Sugiyama, M. et al., Gastroenterology 114: 550-558, (1998)). It is therefore desirable to develop modulators (e.g., antagonists) to signaling pathway components of the TGFβ family to prevent/treat disorders related to the malfunctioning of this signaling pathway.

### SUMMARY OF THE INVENTION

The invention is based on the discovery that compounds of formula (I) are unexpectedly potent antagonists of the TGFβ family type I receptors, Alk5 and/or Alk 4. Thus, compounds of formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

In one aspect, a compound of formula (I):

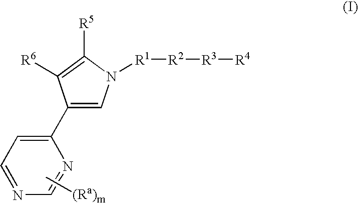

Each R^{a}, independently, can be alkyl, alkenyl, alkynyl, alkoxy, acyl, halo, hydroxy, —NH₂, —NH(unsubstituted alkyl), —N(unsubstituted alkyl)₂, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, cycloalkylcarbonyl, heterocycloalkyl, heterocycloalkyloxy, heterocycloalkylsulfanyl, heterocycloalkylcarbonyl, aryloxy, arylsulfonyl, aroyl, heteroaryl, heteroaryloxy, heteroarylsulfonyl, or heteroaroyl.

R¹ can be a bond, alkylene, alkenylene, alkynylene, or —(CH₂)ᵣ₁—O—(CH₂)ᵣ₂—, where each of r1 and r2, independently, is 2 or 3.

R² can be cycloalkylene, heterocycloalkylene, cycloalkenylene, heterocycloalkenylene, arylene, heteroarylene, or a bond.

R³ can be —C(O)—, —C(O)—O—, —O—C(O)—, —S(O)ₚ—O—, —O—S(O)ₚ—, —C(O)—N(R^{b})—, —N(R^{b})—C(O)—, —O—C(O)—N(R^{b})—, —N(R^{b})—C(O)—O—, —C(O)—N(R^{b})—O—, —O—N(R^{b})—C(O)—, —O—S(O)—N(R^{b})—, —N(R^{b})—S(O)ₚ—O—, —S(O)ₚ—N(R^{b})—O—, —O—N(R^{b})—S(O)ₚ—, —N(R^{b})—C(O)—N(R^{c})—, —N(R^{b})—S(O)ₚ—N(R^{c})—, —C(O)—N(R^{b})—S(O)ₚ—, —S(O)ₚ—N(R^{b})—C(O)—, —C(O)—N(R^{b})—S(O)ₚ—N(R^{c})—, —C(O)—O—S(O)ₚ—N(R^{b})—, —N(R^{b})—S(O)ₚ—N(R^{c})—C(O)— —N(R^{b})—S(O)ₚ—O—C(O)—, —S(O)ₚ—N(R^{b})—, —N(R^{b})—S(O)ₚ—, —N(R^{b})—, —S(O)ₚ—, —O—, —S—, —(C(R^{b})(R^{c}))_{q}—, or a bond. Each of R^{b} and R^{c}, independently, can be hydrogen, hydroxy, alkyl, aryl, aralkyl, heterocycloalkyl, heteroaryl, or heteroaralkyl. p can be 1 or 2 and q can be 1-4.

R⁴ can be hydrogen, alkyl, alkenyl, alkynyl, cycloalkyl, (cycloalkyl)alkyl, heterocycloalkyl, (heterocycloalkyl)alkyl, cycloalkenyl, (cycloalkenyl)alkyl, heterocycloalkenyl, (heterocycloalkenyl)alkyl, aryl, aralkyl, heteroaryl, or heteroaralkyl.

R⁵ can be hydrogen, unsubstituted alkyl, halo-substituted alkyl, alkoxy, alkylsulfinyl, amino, alkenyl, alkynyl, cycloalkoxy, cycloalkylsulfinyl, heterocycloalkoxy, heterocycloalkylsulfinyl, aryloxy, arylsulfinyl, heteroaryloxy, or heteroarylsulfinyl.

R⁶ can be a 5- to 6-membered monocyclic heterocyclyl or a 8- to 11-membered bicyclic heteroaryl. Each can be optionally substituted with alkyl, alkenyl, alkynyl, alkoxy, acyl, halo, hydroxy, amino, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, heterocycloalkyl, heterocycloalkyloxy, heterocycloalkylsulfanyl, cycloalkylcarbonyl, heterocycloalkylcarbonyl, aryl, aryloxy, arylsulfonyl, aroyl, heteroaryl, heteroaryloxy, heteroarylsulfonyl, or heteroaroyl.

The value of m is 0-3, provided that when m≧2, two adjacent R^{a} groups can join together to form a 4- to 8-membered optionally substituted cyclic moiety.

In one embodiment, R⁶ is a 5- to 6-membered heterocyclyl containing 1-3 hetero ring atoms. The hetero ring atoms can be —O—, —S—, —N═, or —NR^{d}—. R^{d} can be hydrogen, alkyl, cycloalkyl, cycloalkylalkyl, aryl, aralkyl, heterocycloalkyl, heterocycloalkylalkyl, heteroaryl, or heteroaralkyl. The heterocyclyl can be optionally substituted with one to two R^{f}, where each R^{f} is independently alkyl, alkenyl, alkynyl, alkoxy, acyl, halo, hydroxy, amino, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, cycloalkylcarbonyl, heterocycloalkyl, heterocycloalkyloxy, heterocycloalkylsulfanyl, heterocycloalkylcarbonyl, aryl, aryloxy, arylsulfonyl, aroyl, heteroaryl, heteroaryloxy, heteroarylsulfonyl, or heteroaroyl. In some circumstances, R^{d} is hydrogen or alkyl. In other circumstances, R⁶ can be a 6-membered heteroaryl containing 1 or 2 hetero ring atoms where each hetero ring atom is —N═ or —NR^{d}—. R⁶ can be

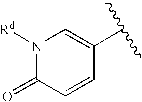

or

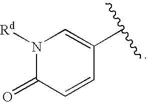

In one embodiment, R⁶ is a fused ring heteroaryl having the formula:

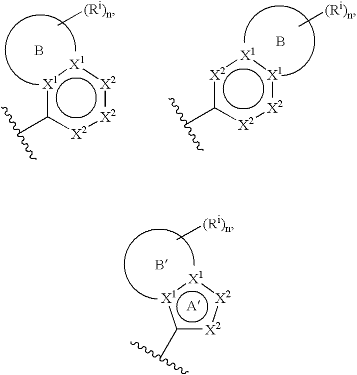

or

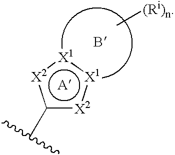

Ring A can be an aromatic ring containing 0-4 hetero ring atoms, and ring B can be a 5- to 7-membered aromatic or nonaromatic ring containing 0-4 hetero ring atoms. At least one of ring A and ring B contains one or more hetero ring atoms. Ring A′ can be an aromatic ring containing 0-4 hetero ring atoms, and ring B′ can be a 5- to 7-membered saturated or unsaturated ring containing 0-4 hetero ring atoms. At least one of ring A′ and ring B′ contains one or more hetero ring atoms. Each hetero ring atom can be —O—, —S—, —N═, or —NR^{g}—. Each X¹ can be independently N or C, and each X² can be independently —O—, —S—, —N═, —NR^{g}—, or —CHR^{h}—. R^{g} can be hydrogen, alkyl, cycloalkyl, cycloalkylalkyl, aryl, aralkyl, heterocycloalkyl, heterocycloalkylalkyl, heteroaryl, or heteroaralkyl. Each of R^{h} and Rⁱ can independently be hydrogen, alkyl, alkenyl, alkynyl, alkoxy, acyl, halo, hydroxy, amino, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, cycloalkylcarbonyl, heterocycloalkyl, heterocycloalkyloxy, heterocycloalkylsulfanyl, heterocycloalkylcarbonyl, aryl, aryloxy, arylsulfonyl, aroyl, heteroaryl, heteroaryloxy, heteroarylsulfonyl, or heteroaroyl; and n is 0-2.

In certain circumstances, R⁶ can be

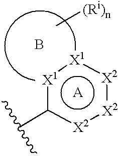

or

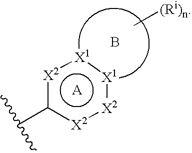

Ring B can be a 5- to 6-membered aromatic or nonaromatic ring. R⁶ can contain at least two hetero ring atoms. R⁶ can contain at least three hetero ring atoms. The para-position of ring A can be occupied by or substituted with one of said hetero ring atoms. Alternatively, the para-position of ring A can be substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—CO—N(R^{j})₂. Each R^{j} can independently be hydrogen, alkyl, cycloalkyl, cycloalkylalkyl, aryl, aralkyl, heterocycloalkyl, heterocycloalkylalkyl, heteroaryl, or heteroaralkyl.

R⁶ can be

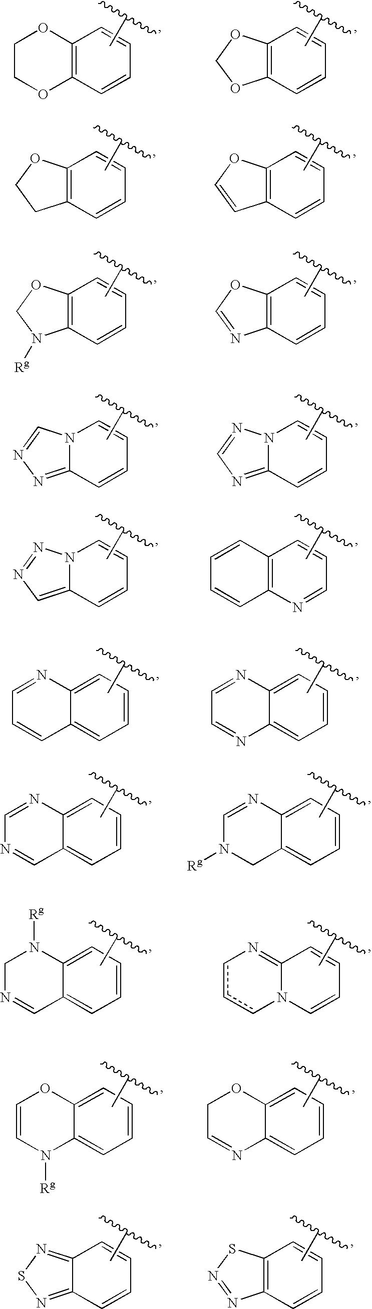

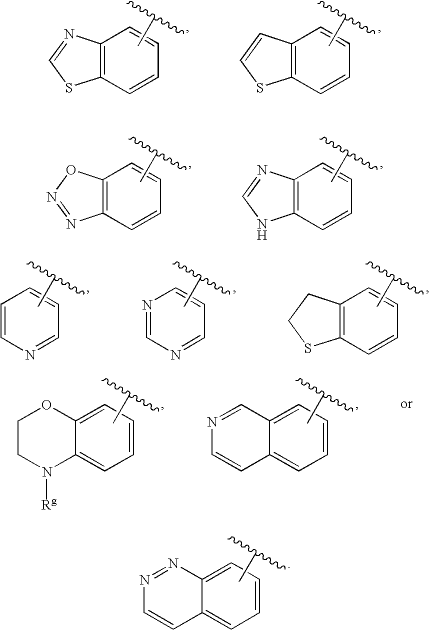

or

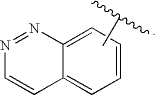

Each of these can be optionally substituted with alkyl, alkoxy, halo, oxo, thioxo, amino, alkylsulfinyl, cyano, carboxy, aryl, or heteroaryl. R^{g} can be hydrogen, alkyl, cycloalkyl, cycloalkylalkyl, aryl, aralkyl, heterocycloalkyl, heterocycloalkylalkyl, heteroaryl, or heteroaralkyl.

R⁶ can be

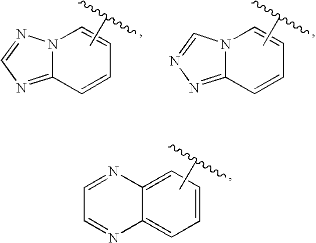

or

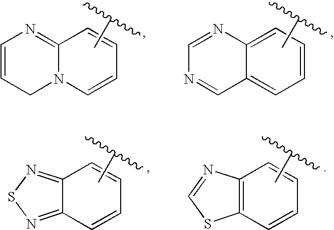

### R⁶ can be

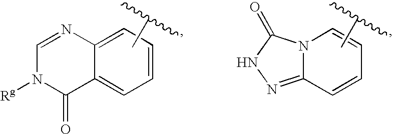

or

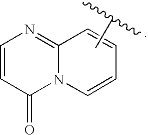

### R⁶ can be

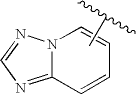

or

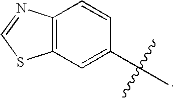

In certain circumstances,

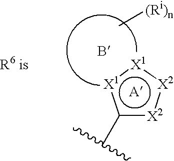

or

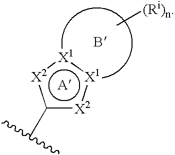

Ring B′ can be a 5- to 6-membered aromatic or nonaromatic ring. R⁶ can contain at least two hetero ring atoms. R⁶ can contain at least three hetero ring atoms.

R⁶ can be

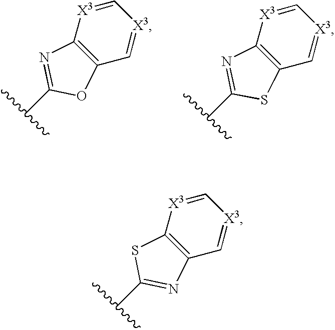

or

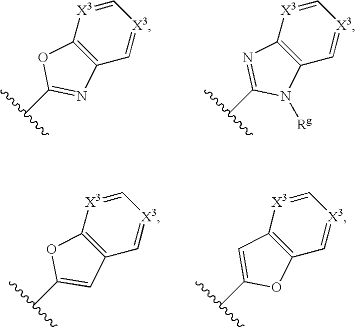

where X³ is independently N or C, and each R⁶ is optionally substituted with alkyl, alkoxy, halo, oxo, thioxo, amino, alkylsulfinyl, cyano, carboxy, aryl, or heteroaryl.

In one embodiment, R¹ is a bond, alkylene, or —(CH₂)₂—O—(CH₂)₂—.

In one embodiment, R¹ is cycloalkylene, heterocycloalkylene, arylene, heteroarylene, or a bond.

In one embodiment, R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, —N(R^{b})—C(O)—O—, —C(O)—N(R^{b})—O—, —N(R^{b})—C(O)—N(R^{c})—, —C(O)—N(R^{b})—S(O)ₚ—N(R^{c})—, —C(O)—O—S(O)ₚ—N(R^{b})—, or a bond.

In one embodiment, R⁴ is hydrogen, alkyl, cycloalkyl, heterocycloalkyl, aryl, or heteroaryl.

When R¹ is a bond or alkylene; R² can be a bond; R³ can be —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S(O)ₚ—N(R^{b})—, N(R^{b})—, or a bond; and R⁴ can be hydrogen, alkyl, cycloalkyl, heterocycloalkyl, aryl, or heteroaryl.

When R¹ is a bond or alkylene; R² can be a bond; R³ can be —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S(O)ₚ—N(R^{b})—, —N(R^{b})—, or a bond; and R⁴ can be hydrogen, alkyl, cycloalkyl, or heterocycloalkyl.

When R¹ is —(CH₂)₂—O—(CH₂)₂—; R² can be piperidinylene, piperazinylene, pyrrolidinylene, tetrahydrofuranylene, tetrahydropyranyl, tetrahydrothiopyranyl, tetrahydrothiopyranyl-1-oxide, tetrahydrothiopyranylene-1-dioxide, cyclohexylene, cyclopentylene, bicyclo[2.2.1]heptanylene, bicyclo[2.2.2]octanylene, bicyclo[3.2.1]octanylene, 2-oxa-bicyclo[2.2.2]octanylene, 2-aza-bicyclo[2.2.2]octanylene, 3-aza-bicyclo[3.2.1]octanylene, cubanylene, or 1-aza-bicyclo[2.2.2]octanylene; R³ can be a bond; and R⁴ can be hydrogen, alkyl, cycloalkyl, heterocycloalkyl, aryl, or heteroaryl.

When R¹ is a bond; R¹ can be piperidinylene, piperazinylene, pyrrolidinylene, tetrahydrofuranylene, tetrahydropyranylene, tetrahydrothiopyranylene, tetrahydrothiopyranylene-1-oxide, tetrahydrothiopyranylene-1-dioxide, cyclohexylene, cyclopentylene, bicyclo[2.2.1]heptanylene, bicyclo[2.2.2]octanylene, bicyclo[3.2.1]octanylene, 2-oxa-bicyclo[2.2.2]octanylene, 2-aza-bicyclo[2.2.2]octanylene, 3-aza-bicyclo[3.2.1]octanylene, cubanylene, or 1-aza-bicyclo[2.2.2]octanylene; R¹ can be —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, or a bond; and R⁴ can be hydrogen, alkyl, cycloalkyl, heterocycloalkyl, aryl, or heteroaryl.

In one embodiment, each of R¹, R², and R³ is a bond; and R⁴ can be hydrogen.

In one embodiment, each of R¹ and R³ is a bond; R² is cycloalkylene, heterocycloalkylene, or a bond; and R⁴ is hydrogen, cycloalkyl, or heterocycloalkyl. For example, -R¹-R²-R³-R⁴ can be

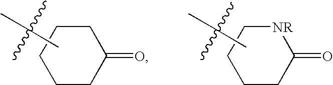

(where R has the same meaning as R^{a}),

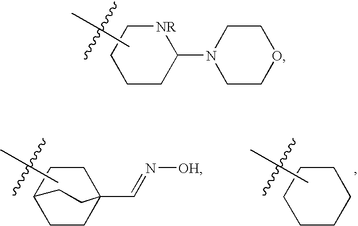

or

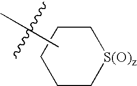

(where z is 0, 1, or 2).

In one embodiment, R⁵ can be hydrogen, unsubstituted alkyl, or halo-substituted alkyl. R⁵ can be hydrogen.

In one embodiment, m is 0, 1, or 2.

In one embodiment, m is 1 or 2 and at least one R^{a} is substituted at the 2-pyrimidinyl position (i.e., the ring position between the two nitrogen ring atoms).

In one embodiment, each R^{a} is independently alkyl, alkoxy, alkylsulfinyl, halo, amino, aminocarbonyl, alkoxycarbonyl, cycloalkyl, or heterocycloalkyl.

In one embodiment, each R^{a} is independently unsubstituted alkyl, halo-substituted alkyl, C₃₋₆ cycloalkyl, or 3- to 6-membered heterocycloalkyl.

In one embodiment, R⁶ is

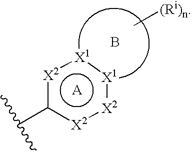

Ring B can be a 5- to 6-membered aromatic or nonaromatic ring. R⁵ can be hydrogen, unsubstituted alkyl, or halo-substituted alkyl. R⁴ can be hydrogen, alkyl, heterocycloalkyl, aryl, or heteroaryl. R³ can be —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, or a bond. R² can be a bond and R¹ can be a bond or alkylene. R^{a} can be alkyl, cycloalkyl, or heterocycloalkyl. If m is not 0, at least one R^{a} is substituted at the position in between the two nitrogen ring atoms.

The para-position of ring A can be occupied by or substituted with a hetero ring atom or the para-position of ring A is substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—CO—N(R^{j})₂. Each R^{j} is independently hydrogen, alkyl, cycloalkyl, cycloalkylalkyl, aryl, aralkyl, heterocycloalkyl, heterocycloalkylalkyl, heteroaryl, or heteroaralkyl.

R⁶ can be

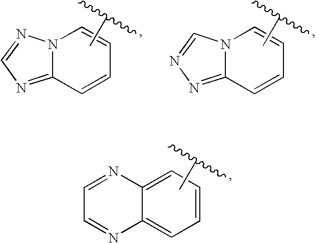

or

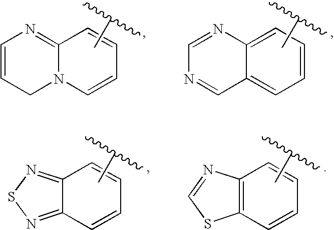

Each of these can be optionally substituted with alkyl, alkoxy, halo, hydroxy, oxo, amino, alkylsulfinyl, cyano, carboxy, aryl, or heteroaryl.

R⁶ can be or each

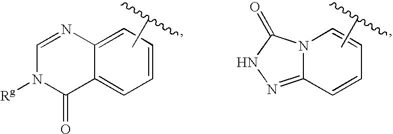

or

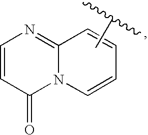

each of which being optionally substituted with alkyl, alkoxy, halo, hydroxy, oxo, amino, alkylsulfinyl, cyano, carboxy, aryl, or heteroaryl.

R⁴ can be hydrogen or alkyl; R³ is —N(R^{b})—C(O)—, —N(R^{b})—S(O)ₚ—, —C(O)—N(R^{b})—, —S(O)ₚ—N(R^{b})—, —N(R^{b})—, or a bond; R² is cycloalkylene or a bond; R¹ is a bond, alkylene, or —(CH₂)₂—O—(CH₂)₂—. R⁴-R³-R¹- can be hydrogen.

R⁵ can be hydrogen, unsubstituted methyl, or trifluoromethyl. R⁵ can be hydrogen.

In certain circumstances, the compound can be 4-(4-benzo[1,3]dioxol-5-yl-1H-pyrazol-3-yl)-2-methyl-pyrimidine, 6-[3-(2-methyl-pyrimidin-4-yl)-1H-pyrazol-4-yl]-[1,2,4]triazolo[1,5-a]pyridine, 6-[3-(2-trifluoromethyl-pyrimidin-4-yl)-1H-pyrazol-4-yl]-[1,2,4]triazolo[1,5-a]pyridine, 6-[3-(2-methyl-pyrimidin-4-yl)-1H-pyrazol-4-yl]-quinoxaline, 6-[3-(2-trifluoromethyl-pyrimidin-4-yl)-1H-pyrazol-4-yl]-quinoxaline, 6-[3-(2-cyclopropyl-pyrimidin-4-yl)-1H-pyrazol-4-yl]-quinoxaline, 4-(4-benzo[1,3]dioxol-5-yl-1H-pyrazol-3-yl)-2-trifluoromethyl-pyrimidine, 7-[3-(2-trifluoromethyl-pyrimidin-4-yl)-1H-pyrazol-4-yl]-[1,2,4]triazolo[1,5-a]pyridine, or 6-[3-(2-Trifluoromethyl-pyrimidin-4-yl)-1H-pyrazol-4-yl]-quinoline.

The compound can be: 6-[3-(2-methyl-pyrimidin-4-yl)-1H-pyrazol-4-yl]-quinoxaline, 6-[3-(2-methyl-pyrimidin-4-yl)-1H-pyrazol-4-yl]-[1,2,4]triazolo[1,5-a]pyridine, and 4-(4-benzo[1,3]dioxol-5-yl-1H-pyrazol-3-yl)-2-methyl-pyrimidine.

In another aspect, a pharmaceutical composition includes a compound of formula (I) and a pharmaceutically acceptable carrier.

In another aspect, a method of inhibiting the TGFβ signaling pathway in a subject, includes administering to the subject with an effective amount of a compound of formula (I).

In another aspect, a method of inhibiting the TGFβ type I receptor in a cell includes the step of contacting said cell with an effective amount of a compound of formula (I).

In another aspect, a method of reducing the accumulation of excess extracellular matrix induced by TGFβ in a subject includes administering to said subject an effective amount of a compound of formula (I).

In another aspect, a method of treating or preventing fibrotic condition in a subject includes administering to said subject an effective amount of a compound of formula (I). The fibrotic condition can be, for example, scleroderma, lupus nephritis, connective tissue disease, wound healing, surgical scarring, spinal cord injury, CNS scarring, acute lung injury, pulmonary fibrosis (such as idiopathic pulmonary fibrosis), chronic obstructive pulmonary disease, adult respiratory distress syndrome, drug-induced lung injury, glomerulonephritis, diabetic nephropathy, hypertension-induced nephropathy, alimentary track or gastrointestinal fibrosis, renal fibrosis, hepatic or biliary fibrosis (such as liver cirrhosis, primary biliary cirrhosis, fatty liver disease, primary sclerosing cholangitis), restenosis, cardiac fibrosis, ophthalmic scarring, fibrosclerosis, fibrotic cancers, fibroids, fibroma, fibroadenomas, fibrosarcomas, transplant arteriopathy, or keloid. The fibrotic condition can be idiopathic in nature, genetically linked, or induced by radiation.

In another aspect, a method of inhibiting growth or metastasis of tumor cells and/or cancers in a subject, includes administering to said subject an effective amount of a compound of formula (I).

In another aspect, a method of treating a disease or disorder mediated by an overexpression of TGFβ includes administering to a subject in need of such treatment an effective amount of a compound of formula (I). The disease or disorder can be, for example, demyelination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, or carcinomas of the lung, breast, ovary, cervix, liver, biliary tract, gastrointestinal tract, pancreas, prostate, and head and neck.

It should be noted that the present invention includes compounds having any combination of the groups described herein.

An N-oxide derivative or a pharmaceutically acceptable salt of each of the compounds of formula (I) is also within the scope of this invention. For example, a nitrogen ring atom of the pyrazole core ring or a nitrogen-containing heterocyclyl substituent can form an oxide in the presence of a suitable oxidizing agent such as m-chloroperbenzoic acid or H₂O₂.

A compound of formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglucamine. A compound of formula (I) can be treated with an acid to form acid addition salts. Examples of such an acid include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to a skilled person in the art. The acid addition salts can be prepared by treating a compound of formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of formula (I) can also be, e.g., in a form of achiral compounds, racemic mixtures, optically active compounds, pure diastereomers, or a mixture of diastereomers.

Compounds of formula (I) exhibit surprisingly high affinity to the TGFβ family type I receptors, Alk 5 and/or Alk 4, e.g., with IC₅₀ and Kᵢ value each of less than 10 μM under conditions as described in Example 10 and Example 12, respectively. Some compounds of formula (I) exhibit IC₅₀ and/or Kᵢ value of below 1.0 μM (or even below 0.1 μM).

Compounds of formula (I) can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

In another aspect, the present invention features a pharmaceutical composition comprising a compound of formula (I) (or a combination of two or more compounds of formula (I)) and a pharmaceutically acceptable carrier. Also included in the present invention is a medicament composition including any of the compounds of formula (I), alone or in a combination, together with a suitable excipient.

In a further aspect, the invention features a method of inhibiting the TGFβ family type I receptors, Alk 5 and/or Alk 4 (e.g., with an IC₅₀ value of less than 10 μM; preferably, less than 1.0 μM; more preferably, less than 0.1 μM) in a cell, including the step of contacting the cell with an effective amount of one or more compounds of formula (I). Also with the scope of the invention is a method of inhibiting the TGFβ and/or activin signaling pathway in a cell or in a subject (e.g., a mammal such as human), including the step of contacting the cell with or administering to the subject an effective amount of one or more of a compound of formula (I).

Also within the scope of the present invention is a method of treating a subject or preventing a subject from suffering a condition characterized by or resulted from an elevated level of TGFβ and/or activin activity. The method includes the step of administering to the subject an effective amount of one or more of a compound of formula (I). The conditions include an accumulation of excess extracellular matrix; a fibrotic condition (which can be induced by drug or radiation), e.g., scleroderma, lupus nephritis, connective tissue disease, wound healing, surgical scarring, spinal cord injury, CNS scarring, acute lung injury, pulmonary fibrosis (such as idiopathic pulmonary fibrosis and radiation-induced pulmonary fibrosis), chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, cirrhosis due to fatty liver disease (alcoholic and nonalcoholic steatosis), primary sclerosing cholangitis, restenosis, cardiac fibrosis, ophthalmic scarring, fibrosclerosis, fibrotic cancers, fibroids, fibroma, fibroadenomas, fibrosarcomas, transplant arteriopathy, and keloid); TGFβ-induced growth or metastasis of tumor/cancer cells; and carcinomas (e.g., squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, and head and neck); and other conditions such as cachexia, hypertension, ankylosing spondylitis, demyelination in multiple sclerosis, cerebral angiopathy and Alzheimer's disease.

As used herein, an “alkyl” group refers to a saturated aliphatic hydrocarbon group containing 1-8 (e.g., 1-6 or 1-4) carbon atoms. An alkyl group can be straight or branched. Examples of an alkyl group include, but are not limited to, methyl, ethyl, propyl, isopropyl, butyl, isobutyl, sec-butyl, tert-butyl, n-pentyl, n-heptyl, and 2-ethylhexyl. An alkyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkylene” is a divalent alkyl group, as defined herein.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkenylene” is a divalent alkenyl group, as defined herein.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkynylene” is a divalent alkynyl group, as defined herein.

As used herein, an “amino” group refers to —NR^{X}R^{Y} wherein each of R^{X} and R^{Y} is independently hydrogen, alkyl, cycloalkyl, (cycloalkyl)alkyl, aryl, aralkyl, heterocycloalkyl, (heterocycloalkyl)alkyl, heteroaryl, or heteroaralkyl. When the term “amino” is not the terminal group (e.g., alkylcarbonylamino), it is represented by —NR^{X}-. R^{X} has the same meaning as defined above.

As used herein, an “aryl” group refers to phenyl, naphthyl, or a benzofused group having 2 to 3 rings. For example, a benzofused group includes phenyl fused with one or two C₄₋₈ carbocyclic moieties, e.g., 1,2,3,4-tetrahydronaphthyl, indanyl, or fluorenyl. An aryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, aldehyde oxime, mercapto, alkylsulfanyl, sulfoxy, urea, thiourea, sulfamoyl, sulfamide, oxo, or carbamoyl. An “arylene” is a divalent aryl group, as defined herein.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl.

As used herein, a “cycloalkyl” group refers to an aliphatic carbocyclic ring of 3-10 (e.g., 4-8) carbon atoms. Examples of cycloalkyl groups include cyclopropyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, and bicyclo[3.2.3]nonyl. A “cycloalkenyl” group, as used herein, refers to a non-aromatic carbocyclic ring of 3-10 (e.g., 4-8) carbon atoms having one or more double bond. Examples of cycloalkenyl groups include cyclopentenyl, 1,4-cyclohexa-di-enyl, cycloheptenyl, cyclooctenyl, hexahydro-indenyl, octahydro-naphthyl, bicyclo[2.2.2]octenyl, and bicyclo[3.3.1]nonenyl. A cycloalkyl or cycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, aldehyde oxime, mercapto, alkylsulfanyl, sulfoxy, urea, thiourea, sulfamoyl, sulfamide, oxo, or carbamoyl. A “cycloalkylene” and a “cycloalkenylene” are a divalent cycloalkyl and a divalent cycloalkenyl group, respectively, as defined herein.

As used herein, a “heterocycloalkyl” group refers to a 3- to 10-membered (e.g., 4- to 8-membered) saturated ring structure, in which one or more of the ring atoms is a heteroatom, e.g., N, O, or S. Examples of a heterocycloalkyl group include piperidinyl, piperazinyl, tetrahydropyranyl, tetrahydrothiopyranyl, tetrahydrothiopyran-1-oxide, tetrahydrothiopyran-1-dioxide, tetrahydrofuryl, dioxolanyl, oxazolidinyl, isoxazolidinyl, morpholinyl, octahydro-benzofuryl, octahydro-chromenyl, octahydro-thiochromenyl, octahydro-indolyl, octahydro-pyrindinyl, decahydro-quinolinyl, octahydro-benzo[b]thiophenyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A “heterocycloalkenyl” group, as used herein, refers to a 3- to 10-membered (e.g., 4- to 8-membered) non-aromatic ring structure having one or more double bonds, and wherein one or more of the ring atoms is a heteroatom, e.g., N, O, or S. A heterocycloalkyl or heterocycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, aldehyde oxime, mercapto, alkylsulfanyl, sulfoxy, urea, thiourea, sulfamoyl, sulfamide, oxo, or carbamoyl. A “heterocycloalkylene” and a “heterocycloalkenylene” are a divalent heterocycloalkyl and a divalent heterocycloalkenyl group, respectively, as defined herein.

A “heteroaryl” group, as used herein, refers to a monocyclic, bicyclic, or tricyclic ring structure having 5 to 15 ring atoms wherein one or more of the ring atoms is a heteroatom, e.g., N, O, or S and wherein one or more rings of the bicyclic or tricyclic ring structure is aromatic. Some examples of heteroaryl are pyridyl, furyl, pyrrolyl, thienyl, thiazolyl, oxazolyl, imidazolyl, indolyl, tetrazolyl, benzofuryl, benzthiazolyl, xanthene, thioxanthene, phenothiazine, dihydroindole, and benzo[1,3]dioxole. A heteroaryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, aldehyde oxime, mercapto, alkylsulfanyl, sulfoxy, urea, thiourea, sulfamoyl, sulfamide, oxo, or carbamoyl. A “heteroarylene” is a divalent heteroaryl, as defined herein. A “heteroaralkyl” group, as used herein, refers to an alkyl group (e.g., a C₁₋₄ alkyl group) that is substituted with a heteroaryl group. Both “alkyl” and “heteroaryl” have been defined above.

As used herein, “cyclic moiety” includes cycloalkyl, heterocycloalkyl, cycloalkenyl, heterocycloalkenyl, aryl, or heteroaryl, each of which has been defined previously.

As used herein, a “hetero ring atom” is a non-carbon ring atom of a heterocycloalkyl, heterocycloalkenyl, or heteroaryl and is selected from the group consisting of oxygen, sulfur, and nitrogen.

As used herein, an “acyl” group refers to a formyl group or alkyl-C(═O)— where “alkyl” has been defined previously. Acetyl and pivaloyl are examples of acyl groups.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} is alkyl, cycloalkyl, (cycloalkyl)alkyl, aryl, aralkyl, heterocycloalkyl, (heterocycloalkyl)alkyl, heteroaryl, or heteroaralkyl.

As used herein, a “carboxy” and a “sulfo” group refer to —COOH and —SO₃H, respectively.

As used herein, an “alkoxy” group refers to an alkyl-O— group where “alkyl” has been defined previously.

As used herein, a “sulfoxy” group refers to —O—SO—R^{X} or —SO—R^{X}, where R^{X} has been defined above.

As used herein, a “halogen” or “halo” group refers to fluorine, chlorine, bromine or iodine.

As used herein, a “sulfamoyl” group refers to the structure —SO₂—NR^{X}R^{Y} or —NR^{X}—SO₂—R^{Z} wherein R^{X}, R^{Y}, and R^{Z} have been defined above.

As used herein, a “sulfamide” group refers to the structure —NR^{X}—S(O)₂—NR^{Y}R^{Z} wherein R^{X}, R^{Y}, and R^{Z} have been defined above.

As used herein, a “urea” group refers to the structure —NR^{X}—CO—NR^{Y}R^{Z} and a “thiourea” group refers to the structure —NR^{X}—CS—NR^{Y}R^{Z}. R^{X}, R^{Y}, and R^{Z} have been defined above.

As used herein, an effective amount is defined as the amount which is required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

An antagonist is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

As compounds of formula (I) are antagonists of TGFβ receptor type I (Alk5) and/or activin receptor type I (Alk4), these compounds are useful in inhibiting the consequences of TGFβ and/or activin signal transduction such as the production of extracellular matrix (e.g., collagen and fibronectin), the differentiation of stromal cells to myofibroblasts, and the stimulation of and migration of inflammatory cells. Thus, compounds of formula (I) inhibit pathological inflammatory and fibrotic responses and possess the therapeutical utility of treating and/or preventing disorders or diseases for which reduction of TGFβ and/or activin activity is desirable (e.g., various types of fibrosis or progressive cancers).

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

Other features and advantages of the invention will be apparent from the following detailed description, and from the claims.

### DETAILED DESCRIPTION OF THE INVENTION

In general, the invention features compounds of formula (I), which exhibit surprisingly high affinity for the TGFβ family type I receptors, Alk 5 and/or Alk 4.

### Synthesis of Compounds of Formula (I)

Compounds of formula (I) may be prepared by a number of known methods from commercially available or known starting materials. In one method, a compound of formula (I) are prepared according to Scheme 1 below. Specifically, a pyrimidine of formula (II), which contains a 2-(α, β-unsaturated carbonyl) substituent can cyclize with hydrazine to form a pyrazole core ring to produce a 2-(pyrazol-3-yl)-pyrimidine intermediate (III). Note that the pyrimidine of formula (II) can be prepared by known methods (see, e.g., Jameson, D. and Guise, L. Tetrahedron Letters, 32(18): 1999-2002). The intermediate (III) can be further substituted at the 4-position of the pyrazole core ring with a good leaving group such as halo (e.g., iodo or bromo) by reacting with a halogenation reagent (e.g., bromination reagent such as Br₂ or iodination reagent such as N-iodosuccinimide) to form a 2-(4-halo-pyrazol-3-yl)-pyrimidine (IV). Note that halo is represented by moiety X in Scheme 1. The halo substituent forms an ideal platform for R⁶ substitutions. For example, the iodo substituent can be converted into a boronic acid substituent (see compound (V) below), which can react with a R⁶-halide (VI) (e.g., an aryl halide or a heteroaryl halide) via Suzuki coupling reaction to form a compound of formula (I). Other substitution reactions can also be employed to produce a wide range of compounds of formula (I) (see, e.g., via a reaction between the protected iodinated compound (IVa) and phthalic anhydride to form a di-keto intermediate (VII), which can undergo a cyclization reaction with an R^{g}-substituted hydrazine to form a compound (I); for reference, see J. Med. Chem., 44(16): 2511-2522 (2001). It should be noted that the pyrazole core ring should be properly protected (see, e.g., the N,N-dimethylaminosulfonyl group of compound (IVa)) to eliminate undesired side reactions.

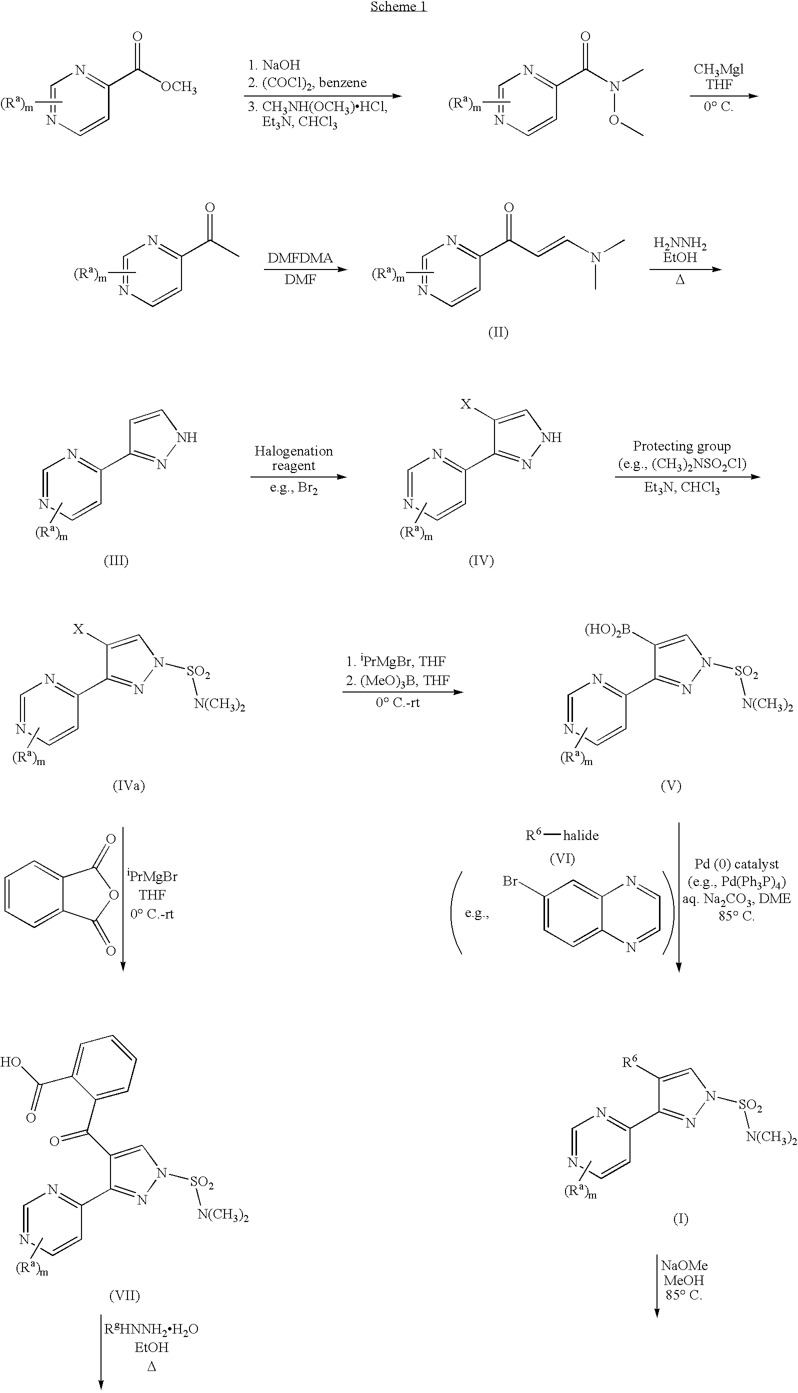

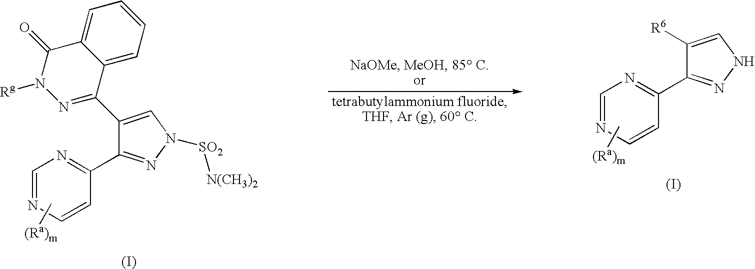

Compounds of formula (VI) are commercially available or can be prepared by known methods. Some exemplary reactions for preparing a compound of formula (VI) are shown below in Scheme 2.

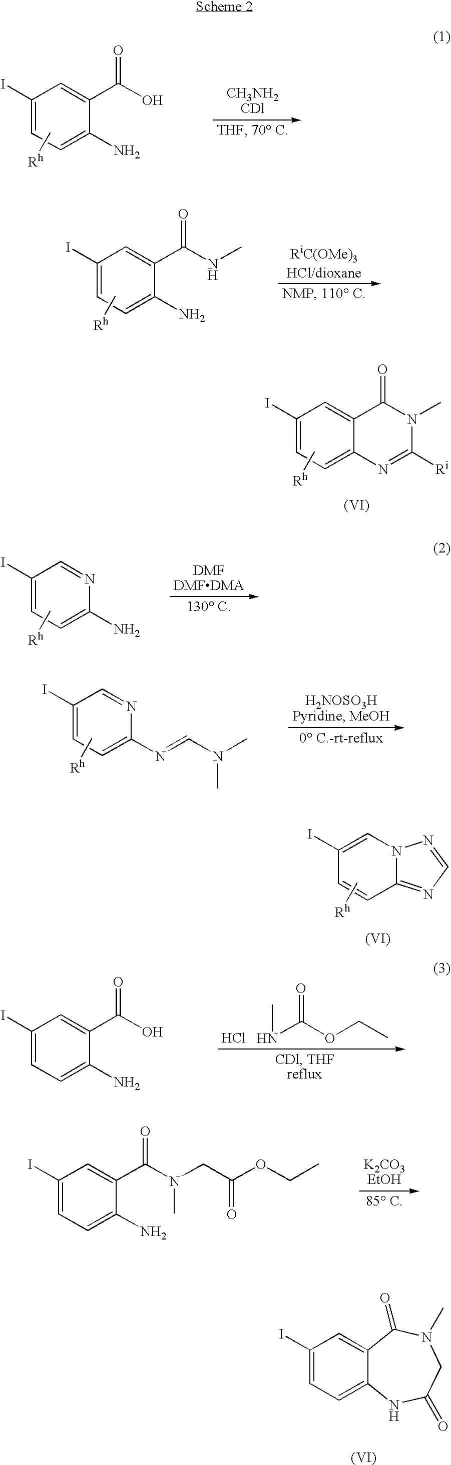

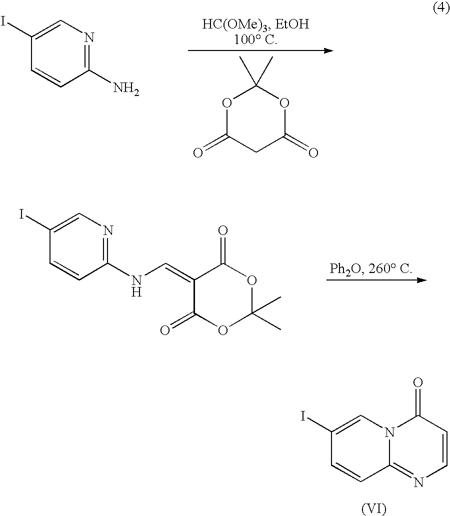

Alternatively, a compound of formula (I) can be prepared according to Scheme 3 below. Specifically, a dimethoxymethyl-substituted pyrimidine of formula (IIa) can be prepared by reacting dimethylformamide dimethylacetal with 1,1-dimethoxy-propan-2-one to form 4-dimethylamino-1,1-dimethoxy-but-3-en-2-one as an intermediate, which can further react with an R^{a}-substituted acetamidine (i.e., R^{a}—C(NH)—NH₂) to produce a compound of formula (IIa). See Reilly, T. A. et al., J. Heterocyclic Chem. 24(4):955 (1989). The compound of formula (IIa) can then be deprotected in an acidic medium (e.g., aqueous HBr) and react with aniline and diphenylphosphite to form a compound of formula (IIb), which can then react with an R⁶-substituted aldehyde to form a compound of formula (IIc). Further reaction of a compound of formula (IIc) with N,N-dimethylformamide dimethylacetal (DMFDMA), followed by hydrazine hydrate, yields a compound of formula (I).

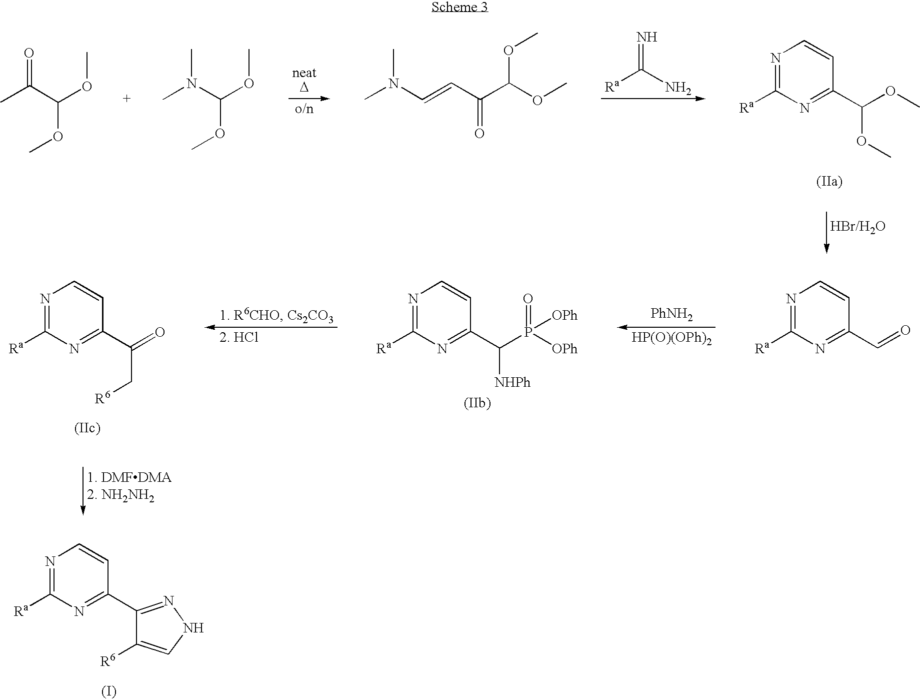

Another method for preparing a compound of formula (I) is shown in Scheme 4 below. Note that R^{a′} has the same meaning as R^{a}, which has been defined above, and X represents halo. Similar to the method described in Scheme 1, this method requires halogenation at the R⁶ position as an intermediate step. See Nesi, R. et al., J. Chem. Soc., Perkin Trans I 8:1667-1770 (1980); Nagamitsu, T. et al. J. Org. Chem. 60(25):8126-8127 (1995); and Guanti, G. and Riva, R. Tetrahedron: Asymmetry 12(8): 1185-1200 (2001) for references for synthesis shown in the first four steps.

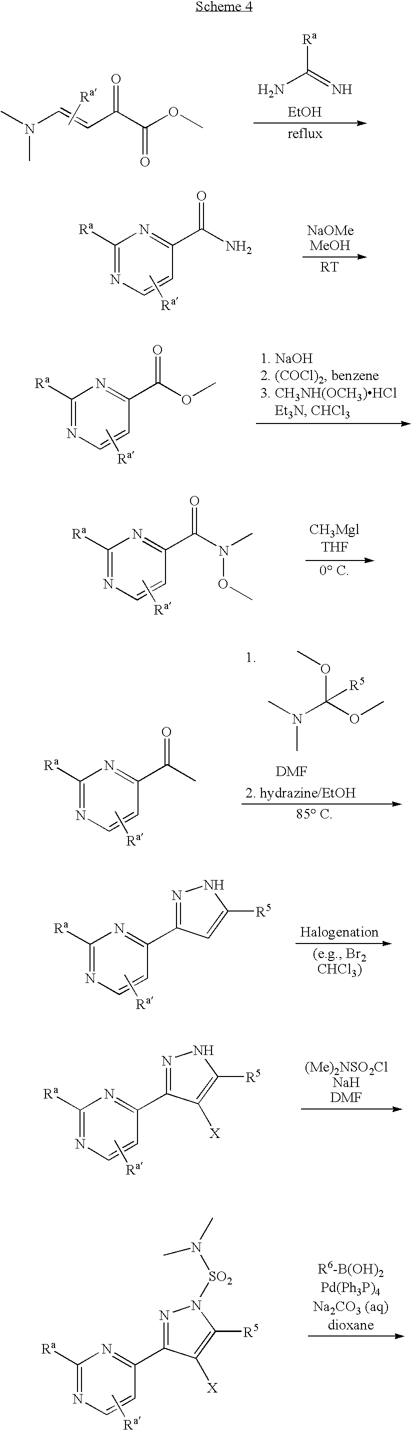

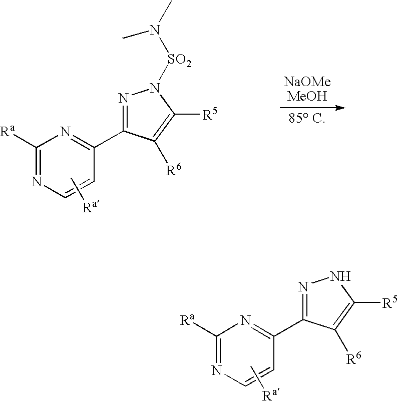

A compound of formula (I) can also be prepared via a phenylacetyl pyrimidine compound (IX) as shown in Scheme 5 below. Specifically, a pyrimidine-carboxyaldehyde compound (VIII) is converted to the N,P acetal intermediate with aniline and diphenylphosphite. This acetal intermediate is then coupled to an aldehyde substituted with R⁶ in basic condition (e.g., Cs₂CO₃) to afford an enamine intermediate, which is hydrolyzed to the ketone intermediate of formula (IX). For reference, see, e.g., Journet et al., Tetrahedron Letters v. 39, p. 1717-1720 (1998). Cyclizing the ketone intermediate (IX) with N,N-dimethylformamide dimethyl acetal and hydrazine affords the pyrazole ring of the desired compound of formula (I). The pyrazole ring of a compound of formula (I) can also be formed by cyclizing the ketone intermediate (IX) with an R⁵-substituted carboxylic acid hydrazide (X). For reference, see, e.g., Chemistry of Heterocyclic compounds 35(11): 1319-1324 (2000).

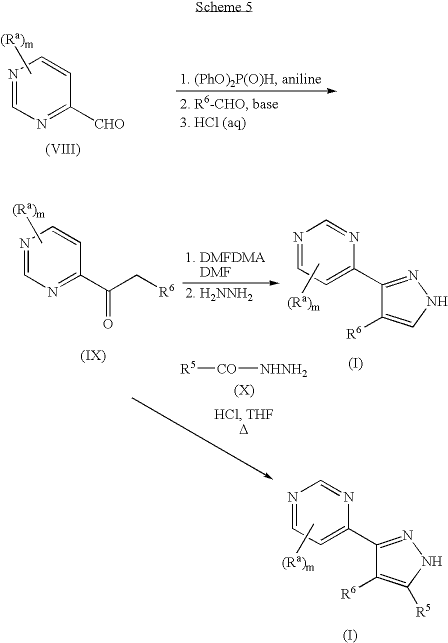

Another method of preparing the intermediate (IX) is depicted in Scheme 6 below. For reference, see, e.g., WO 02/066462, WO 02/062792, and WO 02/062787.

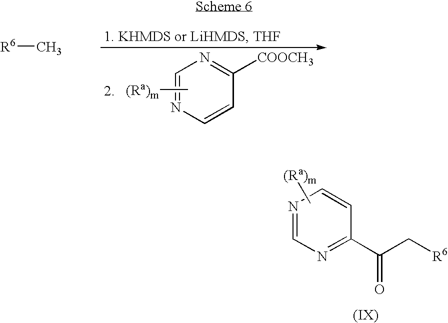

Some methods for preparing a compound of formula (I) wherein -R¹-R²-R³-R⁴ is not hydrogen are shown in Scheme 7 below. In reaction (A) below, a compound of formula (I) wherein the 1-position of the pyrazole core ring is unsubstituted undergoes a substitution reaction with X-R¹-R²-R³-R⁴ where X is a leaving group such as trifluoromethylsulfonate, tosylate, and halide, e.g., Cl, Br, or I. Alternatively, a compound of formula (I) wherein the 1-position of the pyrazole core ring is unsubstituted can undergo a conjugate addition reaction as shown in reaction (B) below. As is well known to a skilled person in the art, the electrophile or acceptor in the addition reaction generally contains a double bond connecting to an electron-withdrawing group or a double bond conjugating to groups such as carbonyl, cyano, or nitro.

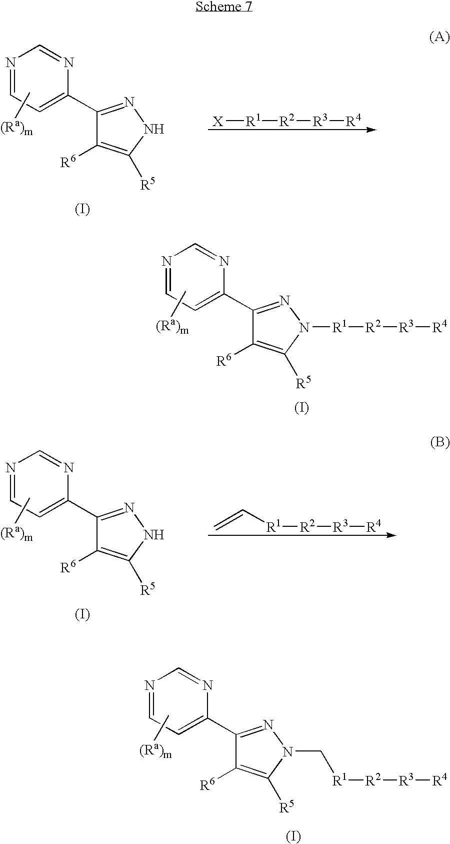

The -R¹-R²-R³-R⁴ group can be further transformed into other functionalities as shown in Scheme 8 below. For example, a compound of formula (I) wherein the -R¹-R²-R³-R⁴ group is cyanoalkyl can be reduced to aminoalkyl, which can be further converted to other functionalities such as heteroaralkyl, heterocycloalkylalkyl, and carboxylic acid.

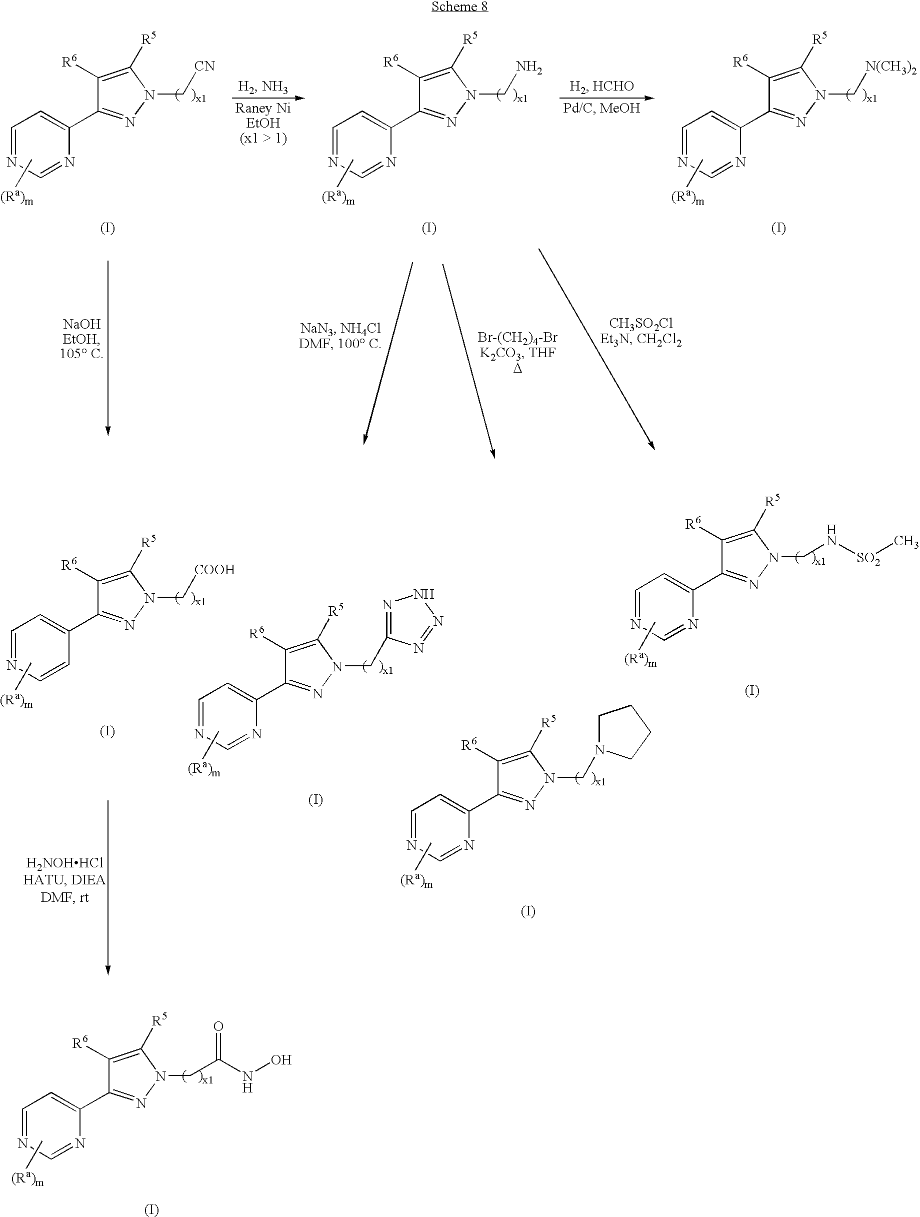

Substituents at the pyrimidinyl ring (i.e., R^{a}) can also be converted into other functionalities. For example, a compound of formula (I) wherein R^{a} is bromo (can be obtained by employing a bromo-substituted compound of formula (VIII) (Sigma-Aldrich, St. Louis, Mo.) can be converted into functionalities such as alkyl, alkenyl, cycloalkyl and the like.

Likewise, substituents of the R⁶ moiety can be further converted into other functionalities as well.

As will be obvious to a skilled person in the art, some starting materials and intermediates may need to be protected before undergoing synthetic steps as described above. For suitable protecting groups, see, e.g., T. W. Greene, Protective Groups in Organic Synthesis, John Wiley & Sons, Inc., New York (1981).

Uses of Compounds of Formula (I)

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Border, W. A. and Ruoslahti E. J. Clin. Invest. 90:1-7 (1992) and Border, W. A. and Noble, N. A. N. Engl. J. Med. 331: 1286-1292 (1994). Studies have been shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis. Elevated TGFβ and/or activin has also been demonstrated in cachexia, demyelination of neurons in multiple sclerosis, Alzheimer's disease, cerebral angiopathy and hypertension.

Compounds of formula (I), which are antagonists of the TGFβ family type I receptors, Alk 5 and/or Alk 4, and inhibit TGFβ and/or activin signaling pathway, are therefore useful for treating and/or preventing disorders or diseases mediated by an increased level of TGFβ and/or activin activity. As used herein, a compound inhibits the TGFβ family signaling pathway when it binds (e.g., with an IC₅₀ value of less than 10 μM; preferably, less than 1 μM; more preferably, less than 0.1 μM) to a receptor of the pathway (e.g., Alk 5 and/or Alk 4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any conditions (a) marked by the presence of an abnormally high level of TGFβ and/or activin; and/or (b) an excess accumulation of extracellular matrix; and/or (c) an increased number and synthetic activity of myofibroblasts. These disorders or diseases include, but are not limited to, fibrotic conditions such as scleroderma, glomerulonephritis, diabetic nephropathy, lupus nephritis, hypertension-induced nephropathy, ocular or corneal scarring, alimentary track or gastrointestinal fibrosis, renal fibrosis, hepatic or biliary fibrosis, acute lung injury, pulmonary fibrosis (such as idiopathic pulmonary fibrosis and radiation-induced pulmonary fibrosis), post-infarction cardiac fibrosis, fibrosclerosis, fibrotic cancers, fibroids, fibroma, fibroadenomas, and fibrosarcomas. Other fibrotic conditions for which preventive treatment with compounds of formula (I) can have therapeutic utility include radiation therapy-induced fibrosis, chemotherapy-induced fibrosis, surgically induced scarring including surgical adhesions, laminectomy, and coronary restenosis.

Increased TGFβ activity is also found to manifest in patients with progressive cancers. Studies have shown that in many cancers, the tumor cells, stromal cells, and/or other cells within a tumor generally overexpress TGFβ. This leads to stimulation of angiogenesis and cell motility, suppression of the immune system, and/or increased interaction of tumor cells with the extracellular matrix. See, e.g., Hojo, M. et al., Nature 397: 530-534 (1999) and Lammerts E. et al., Int. J. Cancer 102: 453-462 (2002). As a result, the tumors grow more readily, become more invasive, and metastasize to distant organs. See, e.g., Maehara, Y. et al., J. Clin. Oncol. 17: 607-614 (1999) and Picon, A. et al., Cancer Epidemiol. Biomarkers Prev. 7: 497-504 (1998). Thus, compounds of formula (I), which are antagonists of the TGFβ type I receptor and inhibit TGFβ signaling pathway, are also useful for treating and/or preventing various cancers which overexpress TGFβ or benefit from TGFβ's above-mentioned pro-tumor activities. Such cancers include carcinomas of the lung, breast, liver, biliary tract, gastrointestinal tract, head and neck, pancreas, prostate, cervix as well as multiple myeloma, melanoma, glioma, and glioblastomas.

Importantly, it should be pointed out that because of the chronic and in some cases localized nature of disorders or diseases mediated by overexpression of TGFβ and/or activin (e.g., fibrosis or cancers), small molecule treatments (such as treatment disclosed in the present invention) are favored for long-term treatment.

Not only are compounds of formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N. Engl. J. Med. 342(18): 1350-1358 (2000); Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); and Sugiyama, M. et al., Gastroenterology 114: 550-558 (1998).

### Administration of Compounds of Formula (I)

As defined above, an effective amount is the amount which is required to confer a therapeutic effect on the treated patient. For a compound of formula (I), an effective amount can range from about 1 mg/kg to about 150 mg/kg (e.g., from about 1 mg/kg to about 100 mg/kg). Effective doses will also vary, as recognized by those skilled in the art, dependant on route of administration, excipient usage, and the possibility of co-usage with other therapeutic treatments including use of other therapeutic agents and/or radiation therapy.

Compounds of formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intramurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, ferret, lizard, reptile, or bird).

Optionally, compounds of formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid and steroid anti-inflammatory agents, immunotherapeutics, chemotherapeutics, as well as agents that antagonize ligand binding or activation of the TGFβ receptors, e.g., anti-TGFβ, anti-TGFβ receptor antibodies, or antagonists of the TGFβ type II receptors. Compounds of formula (I) can also be administered in conjunction with other treatments, e.g., radiation.

The invention will be further described in the following examples, which do not limit the scope of the invention described in the claims.

### EXAMPLE 1

### 4-(4-Benzo[1,3]dioxol-5-yl-1H-pyrazol-3-yl)-2-methyl-pyrimidine

Synthesis of the title compound is described in parts (a)-(c) below.

### (a) (Phenylamino-(2-methyl-pyrimidin-4-yl)-methyl)-phosphonic acid diphenyl ester (1a)

4-Dimethoxymethyl-2-methyl-pyrimidine (4.5 g, 26.7 mmol) was added to a solution of HBr (48% in H₂O, 10 mL) and stirred at room temperature for 2 hours. It was then diluted with water and washed with diethylether (2×). The aqueous layer was carefully neutralized with saturated sodium carbonate and extracted with ethyl acetate (2×). The combined extracts were dried over MgSO₄. 2-Propanol (100 mL) was added. To this solution, aniline (2.5 mL, 26.7 mmol) was added and followed with diphenylphosphite (5.1 mL, 26.7 mmol). The reaction mixture was stirred at room temperature overnight and then concentrated. The residue was purified on silica gel column with 20% ethyl acetate/CH₂Cl₂ to give a yellow solid (3.3 g, 29% for 2 steps) as the desired product. MS (ESP⁺) m/z 432.2 (M+1).

### (b) 2-Benzo[1,3]dioxol-5-yl-1-(2-methyl-pyrimidin-4-yl)-ethanone

To a solution of benzo[1,3]dioxole-5-carbaldehyde (1.89 g, 0.0126 mol; Aldrich) in a mixed solvent of THF (40 mL) and iPrOH (10 mL), was added (phenylamino-(2-methyl-pyrimidin-4-yl)-methyl)-phosphonic acid diphenyl ester (5.46 g, 0.0126 mol) and Cs₂CO₃ (5.39 g, 0.0164 mol). It was stirred at room temperature for 20 hours and then treated with 3N HCl (10 mL) for 1 hour. The reaction mixture was then diluted with methyl t-butyl ether and extracted with 1N HCl twice. The combined aqueous layers were neutralized with 30% aqueous KOH to pH of ca. 8, then extracted with ethyl acetate (3×). Organic layers were dried over MgSO₄ and concentrated to yield a dark orange oil, which was purified on silica gel column with EtOAc/hexane (4:1) to give 2-benzo[1,3]dioxol-5-yl-1-(2-methyl-pyrimidin-4-yl)-ethanone (2.02 g, 60%) as a yellow solid.

### (c) 4-(4-Benzo[1,3]dioxol-5-yl-1H-pyrazol-3-yl)-2-methyl-pyrimidine

To a solution of 2-benzo[1,3]dioxol-5-yl-1-(2-methyl-pyrimidin-4-yl)-ethanone (100 mg, 0.39 mmol) in DMF (3 mL) was added acetic acid (0.054 mL, 0.94 mmol) and dimethylformamide dimethyl acetal (0.26 mL, 1.95 mmol) and stirred at room temperature for 5 hours. Hydrazine hydrate (0.19 mL, 3.9 mmol) was then added and heated to 50° C. for 2 hours until the reaction completed. The reaction mixture was then concentrated and worked up with ethyl acetate and water. The organic layer was dried over MgSO₄ and concentrated. The residue was purified on semi-preparative HPLC to give 4-(4-benzo[1,3]dioxol-5-yl-1H-pyrazol-3-yl)-2-methyl-pyrimidine (30 mg) as a TFA salt. LC-MS/ES+: M+1:281.0. ¹H NMR (300 MHz, MeOH-d4), δ 8.65 (d, 1H), 7.80 (s, 1H), 7.58 (d, 1H), 6.95-6.87 (m, 3H), 6.07 (s, 2H), 2.60 (s, 3H).

### EXAMPLE 2

### 6-[3-(2-Methyl-pyrimidin-4-yl)-1H-pyrazol-4-yl]-[1,2,4]triazolo[1,5-a]pyridine

Synthesis of the title compound is described in parts (a)-(c) below.

### (a) [1,2,4]triazolo[1,5-a]pyridine-6-carbaldehyde

To a solution of 6-iodo[1,2,4]triazolo[1,5-a]pyridine (5 g, 0.02 mol, prepared according to literature procedure) in anhydrous THF (300 mL), was slowly added 1M of isopropylmagnesium bromide in THF (31 mL, 0.03 mol) at 0° C. It was stirred at 0° C. for 1 hour and then was added anhydrous DMF (6 mL, 0.05 mol). It was allowed to warm to room temperature and stirred for overnight. It was then quenched with 100 mL of water and worked up with diethyl ether and saturated NaHCO₃. Dried over MgSO₄ and concentrated. The residue was purified on silica gel column with EtOAc to give the desire product as a tan solid (3 g, 100%). LC-MS/ES+: M+1:148.0.

### (b) 1-(2-methyl-pyrimidin-4-yl)-2-[1,2,4]triazolo[1,5-a]pyridin-6-yl-ethanone

To a solution of [1,2,4]triazolo[1,5-a]pyridine-6-carbaldehyde (2.5 g, 0.0169 mol) in a mixed solvent of THF (40 mL) and iPrOH (10 mL), was added (phenylamino-(2-methyl-pyrimidin-4-yl)-methyl)-phosphonic acid diphenyl ester (7.33 g, 0.0169 mol; see Example 1(b) above) and Cs₂CO₃ (7.26 g, 0.022 mol). It was stirred at room temperature for 48 hours and then treated with 3N HCl (10 mL) for 1 hour. The reaction mixture was then diluted with methyl t-butyl ether and extracted with 1N HCl twice. The combined aqueous layers were neutralized with 30% aqueous KOH to pH of ca. 8, then extracted with ethyl acetate (3×). Organic layers were dried over MgSO₄ and concentrated to yield a dark orange oil, which was purified on silica gel column with EtOAc/hexane (4:1) to give 1-(2-methyl-pyrimidin-4-yl)-2-[1,2,4]triazolo[1,5-a]pyridin-6-yl-ethanone (4.15 g, 97%) as a yellow solid. ¹H NMR (300 MHz, CDCl₃), δ 8.94 (d, 1H), 8.62 (s, 1H), 8.34 (s, 1H), 7.76 (d, 1H), 7.75 (s, 1H), 7.51 (d, 1H), 4.61 (s, 2H), 2.90 (s, 3H).

### (c) 6-[3-(2-Methyl-pyrimidin-4-yl)-1H-pyrazol-4-yl]-[1,2,4]triazolo[1,5-a]pyridine

Acetic acid (0.286 mL, 5 mmol) was added to a solution of 1-(2-methyl-pyrimidin-4-yl)-2-[1,2,4]triazolo[1,5-a]pyridin-6-yl-ethanone (0.253 g, 1 mmol) in DMF (10 mL). The mixture was stirred for five minutes. DMF-DMA (0.668 mL, 5 mmol) was then added. The mixture was stirred for 1 hour. Hydrazine monohydrate (0.484 mL, 10 mmol) was added. The mixture was heated at 50° C. for 3 hours. The mixture was partitioned between ethyl acetate and water. Ethyl acetate was washed with brine, dried over sodium sulfate, filtered, and concentrated. HPLC purification gave 0.06 g (22%) of the title compound as a yellow solid. LC-MS/ES+: M+1:278.3. ¹H NMR (300 MHz, MeOH-d₄): δ 9.12 (s, 1H), 8.72 (d, 1H, J=5.7 Hz), 8.53 (s, 1H), 8.10 (s, 1H), 7.98 (d, 1H, J=5.7 Hz), 7.92 (dd, 1H, J=9.3 Hz, 1.5 Hz), 7.81 (d, 1H, J=9.3 Hz), 2.65 (s, 3H).

### EXAMPLE 3

### 6-[3-(2-Trifluoromethyl-pyrimidin-4-yl)-1H-pyrazol-4-yl]-[1,2,4]triazolo[1,5-a]pyridine

Synthesis of the title compound is described in parts (a)-(d) below.

### (a) 4-Dimethoxymethyl-2-trifluoromethyl-pyrimidine (1b)

Equimolar amounts of dimethylformamide dimethylacetal (15 mL, 0.11 mol) and 1,1-Dimethoxy-propan-2-one (14 mL, 0.11 mol) were combined and heated to 80° C. overnight. After cooling to room temperature, volatile materials were evaporated. 4-Dimethylamino-1,1-dimethoxy-but-3-en-2-one resulted as a dark brown liquid (20 g) without further purifications. ¹H NMR: (300 MHz, CDCl₃), δ 7.70 (d, 1H), 5.30 (d, 1H), 4.54 (s, 1H), 3.37 (s, 6H), 3.08 (s, 3H), 2.83 (s, 3H).

To a solution of the above compound (15 g, 86.6 mmol) in ethanol (50 mL) was added trifluoroacetamidine (10.7 g, 95.2 mmol) and heated to reflux overnight. It was then cooled down to room temperature and concentrated in vacuo. The residue was purified on silica gel column with 100% CH₂Cl₂ to give 4-dimethoxymethyl-2-trifluoromethyl-pyrimidine as a light yellow liquid (8.5 g) as (R_{f}=0.2 using 100% CH₂Cl₂). ¹H NMR: (300 MHz, CDCl₃), δ 8.94 (s, 1H), 7.75 (s, 1H), 5.33 (s, 1H), 3.46 (s, 6H).

### (b) (Phenylamino-(2-trifluoromethyl-pyrimidin-4-yl)-methyl)-phosphonic acid diphenyl ester

4-Dimethoxymethyl-2-trifluoromethyl-pyrimidine (9.4 g, 42.3 mmol) was added to a solution of HBr (48% in H₂O, 10 mL) and stirred at room temperature overnight. It was then diluted with water and washed with diethylether (2×). The aqueous layer was carefully neutralized with saturated potassium hydroxide and extracted with ethyl acetate (2×). The combined extracts were dried over MgSO₄ and 2-propanol (100 mL) was added and followed with addition of aniline (3.9 mL, 42.3 mmol) and diphenylphosphite (8.1 mL, 42.3 mmol). The reaction mixture was stirred at room temperature overnight and then concentrated. The residue was purified on silica gel column with 20% ethyl acetate/CH₂Cl₂ to give a syrup which was crystallized from cold 2-propanol to give (Phenylamino-(2-trifluoromethyl-pyrimidin-4-yl)-methyl)-phosphonic acid diphenyl ester as a white solid (5 g, 24% for 2 steps) as the desired product. MS (ESP⁺) m/z 485.9 (M+1).

### (c) 2-[1,2,4]Triazolo[1,5-a]pyridin-6-yl-1-(2-trifluoromethyl-pyrimidin-4-yl)-ethanone

To a solution of [1,2,4]triazolo[1,5-a]pyridine-6-carbaldehyde (0.91 g, 6.1 mmol; see Example 2a) in a mixed solvent of THF (40 mL) and iPrOH (10 mL), was added (phenylamino-(2-trifluoromethyl-pyrimidin-4-yl)-methyl)-phosphonic acid diphenyl ester (3.0 g, 6.1 mmol) and Cs₂CO₃ (2.64 g, 8 mmol). It was stirred at room temperature for 16 hours and then treated with 3N HCl (10 mL) for 1 hour. The reaction mixture was then diluted with methyl t-butyl ether and extracted with 1N HCl twice. The combined aqueous layers were neutralized with 30% aqueous KOH to pH of ca. 8, and yellow precipitation was collected and dried to give 2-[1,2,4]triazolo[1,5-a]pyridin-6-yl-1-(2-trifluoromethyl-pyrimidin-4-yl)-ethanone (1.08 g, 57%) as a yellow solid. LC-MS/ES+: M+1:308.3. ¹H NMR (300 MHz, CDCl₃), δ 9.38 (s, 1H), 8.89 (d, 1H), 8.59-8.31 (m, 2H), 7.87-7.50 (m, 3H), 6.43 (s, 1H).

### (d) 6-[3-(2-Trifluoromethyl-pyrimidin-4-yl)-1H-pyrazol-4-yl]-[1,2,4]triazolo[1,5-a]-pyridine

Acetic acid (0.286 mL, 5 mmol) was added to a solution of 2-[1,2,4]triazolo[1,5-a]pyridin-6-yl-1-(2-trifluoromethyl-pyrimidin-4-yl)-ethanone (0.307 g, 1.0 mmol) in DMF (10 mL). The mixture was stirred for five minutes. DMF-DMA (0.668 mL, 5 mmol) was then added. The mixture was stirred for 1 hour. Hydrazine monohydrate (0.484 mL, 10 mmol) was added. The mixture was heated at 50° C. for 3 hours. The mixture was partitioned between ethyl acetate and water. Ethyl acetate was washed with brine, dried over sodium sulfate, filtered, and concentrated. HPLC purification gave 0.07 g (21%) of the title compound as a yellow solid. LC-MS/ES+: M+1:332.3. ¹H NMR (300 MHz, DMSO-d₆): δ 9.16 (s, 1H), 9.03 (d, 1H, J=3 Hz), 8.50 (s, 1H), 8.24 (d, 1H, J=6 Hz), 8.17 (d, 1H, J=6 Hz), 7.79 (s, 2H).

### EXAMPLE 4

### 6-[3-(2-Methyl-pyrimidin-4-yl)-1H-pyrazol-4-yl]-quinoxaline

Synthesis of the title compound is described in parts (a)-(c) below.

### (a) Quinoxaline-6-carbaldehyde

6-Methylquinaxoline (100 g, 0.69 mol) was heated in a sealed tube to 160° C. and was then added selenium dioxide (100 g, 0.90 mol). The sealed tube was then stirred at 160° C. for 3 days, then allowed to cool to room temperature. The contents solidified and were dissolved in dichloromethane. Solids were filtered through a celite/silica gel cake. The cake was washed with dichloromethane and washes were combined and concentrated to give a pinkish solid, which was washed with hexane and then dried under vacuum to give quinoxaline-6-carbaldehyde as a white solid (50.5 g, contained ca. 10% of 6-methylquinazoline).

### (b) 1-(2-Methyl-pyrimidin-4-yl)-2-quinoxalin-6-yl-ethanone

To a solution of quinoxaline-6-carbaldehyde (2.0 g, 0.0126 mol) in THF (40 mL) and iPrOH (10 mL), was added (phenylamino-(2-methyl-pyrimidin-4-yl)-methyl)-phosphonic acid diphenyl ester (5.46 g, 0.0126 mol) and Cs₂CO₃ (5.39 g, 0.0164 mol). The mixture was stirred at room temperature for 20 hours and then treated with 3N HCl (10 mL) for 1 hour. The reaction mixture was then diluted with methyl t-butyl ether and extracted with 1N HCl twice. The combined aqueous layers were neutralized with 30% aqueous KOH to a pH of ca. 8, then extracted with ethyl acetate (3×). Organic layers were dried over MgSO₄ and concentrated to yield a dark orange oil, which was purified on silica gel column with ethyl acetate/hexane (4:1) to give 1-(2-methyl-pyrimidin-4-yl)-2-quinoxalin-6-yl-ethanone (2.02 g, 60%) as a yellow solid. LC-MS/ES+: M+1:265.16. ¹H NMR (300 MHz, CDCl₃), δ 8.91 (d, 1H), 8.85 (m, 2H), 8.1 (d, 2H), 7.75 (d, 2H), 4.71 (s, 2H), 2.81 (s, 3H).

### (c) 6-[3-(2-Methyl-pyrimidin-4-yl)-1H-pyrazol-4-yl]-quinoxaline

Acetic acid (0.286 mL, 5 mmol) was added to a solution of 1-(2-methyl-pyrimidin-4-yl)-2-quinoxalin-6-yl-ethanone (0.264 g, 1.0 mmol) in DMF (10 mL). The mixture was stirred for 5 minutes. DMF-DMA (0.668 mL, 5 mmol) was then added. The mixture was stirred for 1 hour. Hydrazine monohydrate (0.484 mL, 10 mmol) was added. The mixture was heated at 50° C. for 3 hours. The mixture was partitioned between ethyl acetate and water. Ethyl acetate was washed with brine, dried over sodium sulfate, filtered, and concentrated. HPLC purification gave 0.23 g (79%) of the title compound as a yellow solid. LC-MS/ES+: M+1:289.29. ¹H NMR (300 MHz, MeOH-d₄): δ 8.88 (m, 2H), 8.69 (m, 1H), 8.18 (m, 1H), 8.08 (m, 2H), 7.96 (m, 1H), 7.83 (m, 1H), 2.56 (s, 3H).

### EXAMPLE 5

### 6-[3-(2-Trifluoromethyl-pyrimidin-4-yl)-1H-pyrazol-4-yl]-quinoxaline

Synthesis of the title compound is described in parts (a) and (b) below.

### (a) 2-Quinoxalin-6-yl-1-(2-trifluoromethyl-pyrimidin-4-yl)-ethanone

To a solution of quinoxaline-6-carbaldehyde (0.96 g, 6.1 mol; see Example 4(a) above) in a mixed solvent of THF (40 mL) and iPrOH (10 mL), was added (phenylamino-(2-trifluoromethyl-pyrimidin-4-yl)-methyl)-phosphonic acid diphenyl ester (3.0 g, 6.1 mmol; see Example 3(b) above) and Cs₂CO₃ (2.64 g, 8 mmol). It was stirred at room temperature overnight and then treated with 3N HCl (10 mL) for 1 hour. The reaction mixture was then diluted with methyl t-butyl ether and extracted with 1N HCl twice. The combined aqueous layers were neutralized with 30% aqueous KOH to pH of ca. 8, then extracted with ethyl acetate (3×). Organic layers were dried over MgSO₄ and concentrated to yield a dark orange oil, which was purified on silica gel column with EtOAc/hexane (4:1) to give 2-quinoxalin-6-yl-1-(2-trifluoromethyl-pyrimidin-4-yl)-ethanone (1.67 g) as a yellow solid. LC-MS/ES+: M+1: 319.39. ¹H NMR (300 MHz, CDCl₃), δ 9.08 (d, 1H), 8.75 (m, 2H), 8.03-7.96 (m, 3H), 7.67 (dd, 1H), 4.71 (s, 2H).

### (b) 6-[3-(2-Trifluoromethyl-pyrimidin-4-yl)-1H-pyrazol-4-yl]-quinoxaline

Acetic acid (0.286 mL, 5 mmol) was added to a solution 2-quinoxalin-6-yl-1-(2-trifluoromethyl-pyrimidin-4-yl)-ethanone (0.318 g, 1.0 mmol) in DMF (10 mL). The mixture was stirred for five minutes. DMF-DMA (0.668 mL, 5 mmol) was then added. The mixture was stirred for 1 hour. Hydrazine monohydrate (0.484 mL, 10 mmol) was added. The mixture was heated at 50° C. for 3 hours. The mixture was partitioned between ethyl acetate and water. Ethyl acetate was washed with brine, dried over sodium sulfate, filtered, and concentrated. HPLC purification gave 0.21 g (61%) of the title compound as a yellow solid. LC-MS/ES+: M+1:343.32. ¹H NMR (300 MHz, MeOH-d₄): δ 8.90 (m, 3H), 8.04 (m, 5H), 2.65 (s, 3H).

### EXAMPLE 6

### 6-[3-(2-Cyclopropyl-pyrimidin-4-yl)-1H-pyrazol-4-yl]-quinoxaline

Synthesis of the title compound is described in parts (a)-(d) below.

### (a) 2-Cyclopropyl-4-dimethoxymethyl-pyrimidine (1c)

The title compound was prepared from cyclopropanecarboxamidine (Lancaster) using the procedure of Example 3(a).

### (b) [(2-Cyclopropyl-pyrimidin-4-yl)-phenylamino-methyl]-phosphonic acid diphenyl ester (2c)

The title compound was prepared according to the procedure of Example 3(b). MS (ESP⁺) m/z 432.2 (M+1).

### (c) 1-(2-Cyclopropyl-pyrimidin-4-yl)-2-quinoxalin-6-yl-ethanone

The title compound was prepared according to the procedure of Example 3(c). LC-MS/ES+: M+1:291.21. ¹H NMR (300 MHz, CDCl₃), δ 8.85 (m, 2H), 8.75 (d, 1H), 8.11 (d, 1H), 8.05 (d, 1H), 7.75 (dd, 1H), 7.65 (d, 1H), 4.63 (s, 2H), 2.42 (m, 1H), 1.30-1.18 (m, 4H).

### (d) 6-[3-(2-Cyclopropyl-pyrimidin-4-yl)-1H-pyrazol-4-yl]-quinoxaline

Acetic acid (0.286 mL, 5 mmol) was added to a solution 1-(2-cyclopropyl-pyrimidin-4-yl)-2-quinoxalin-6-yl-ethanone (0.29 g, 1.0 mmol) in DMF (10 mL). The mixture was stirred for five minutes. DMF-DMA (0.668 mL, 5 mmol) was then added. The mixture was stirred for 1 hour. Hydrazine monohydrate (0.484 mL, 10 mmol) was added. The mixture was heated at 50° C. for 3 hours. The mixture was partitioned between ethyl acetate and water. Ethyl acetate was washed with brine, dried over sodium sulfate, filtered, and concentrated. HPLC purification gave 0.25 g (79%) of the title compound as a yellow solid. LC-MS/ES+: M+1:315.33. ¹H NMR (300 MHz, MeOH-d₄): δ 8.90 (s, 2H), 8.62 (d, 1H, J=6 Hz), 8.11 (m, 2H), 8.04 (s, 1H), 7.90 (m, 2H), 2.66 (s, 3H), 0.85 (m, 2H), 0.64 (m, 2H).

### EXAMPLE 7

### 4-(4-Benzo[1,3]dioxol-5-yl-1H-pyrazol-3-yl)-2-trifluoromethyl-pyrimidine

Synthesis of the title compound is described in parts (a)-(g) below.

### (a) 2-(Trifluoromethyl)-N-methoxy-N-methylpyrimidine-4-carboxamide

To a solution of 4.0 g (19.4 mmol) of 2-trifluoromethyl-pyrimidine-4-carboxylic acid methyl ester (CNH Technologies, Inc., Woburn, Mass.) in a mixture of 50 mL THF and 50 mL methanol was added 2.0 mL (20 mmol) of 10 M NaOH solution with stirring to give a colorless solution. This was stirred at room temperature for two hours, after which LCMS showed no starting material remained, so the solution was concentrated to a white solid. This was suspended in 50 mL benzene with stirring. The mixture was then cooled to 0° C., and 17.2 mL (197 mmol) of oxalyl chloride was added slowly. The resulting mixture was heated to reflux overnight, then cooled and concentrated. The yellow/brown residue was dissolved in 80 mL CH₂Cl₂ to give a yellow solution. To this was added 8.3 mL (60 mmol) of triethylamine, followed by 2.3 grams of N,O-dimethylhydroxylamine hydrochloride to give a brown mixture. This was heated to reflux overnight, then cooled, diluted with additional CH₂Cl₂, washed twice with a 5% solution of citric acid, twice with 1N NaOH, and the organic phase dried (Na₂SO₄), and concentrated to a yellow oil. Purification by flash chromatography (2:1 hexanes/ethyl acetate going to 1:1 hexanes/ethyl acetate vs. silica) gave 3.28 g (13.9 mmol, 72%) of 2-trifluoromethyl-pyrimidine-4-carboxylic acid methoxy-methyl-amide as a pale yellow oil. ¹H-NMR (300 MHz, CDCl₃) δ: 9.05 (d, J=5 Hz, 1H), 7.76 (d, J=5 Hz, 1H), 3.84 (s, 3H), 3.38 (s, 3H); m/z: 236 [M+H]⁺.

### (b) 1-(2-(Trifluoromethyl)-pyrimidin-4-yl)-ethanone

To an ice-cold, stirred solution of 3.28 g (13.9 mmol) of trifluoromethyl-pyrimidine-4-carboxylic acid methoxy-methyl-amide in 30 mL dry DMF under dry nitrogen was added 4.7 mL (14 mmol) of a 3.0 M solution of methylmagnesium iodide in ether dropwise via syringe. The resulting orange solution was warmed to RT over 15 minutes and quenched with 5 mL of a saturated ammonium chloride solution. This mixture was diluted with 100 mL ethyl acetate, washed once with a saturated solution of NaHCO₃, then brine, dried (Na₂SO₄) and concentrated. The residue was purified by flash chromatography (4:1 hexanes/ethyl acetate vs. silica) to give 1.47 g (7.7 mmol, 55%) of 1-(2-trifluoromethyl-pyrimidin-4-yl)-ethanone as a pale yellow liquid. ¹H-NMR (300 MHz, CDCl₃, δ): 9.15 (d, J=5 Hz, 1H), 8.08 (d, J=5 Hz, 1H), 2.78 (s, 3H); ¹³C-NMR (400 MHz, CDCl₃) δ: 197.9, 160.1, 159.4, 120.7, 118.9, 117.9, 25.4; m/z: 191 [M+H]⁺.

### (c) 4-(1H-Pyrazol-3-yl)-2-trifluoromethyl-pyrimidine

To a solution of 1.47 g (7.7 mmol) of 1-(2-trifluoromethyl-pyrimidin-4-yl)-ethanone in 5 mL DMF was added 5.0 mL (excess) of DMF-dimethyl acetal with stirring to give a dark solution. This was heated to 85° C. for 30 minutes, then cooled and concentrated. The dark residue was dissolved in 10 mL EtOH with stirring and 1 mL (excess) of hydrazine hydrate added to give a red solution. This was heated to reflux for 60 minutes, then cooled and concentrated to a red oil. This was purified by flash chromatography (1:1 hexanes/ethyl acetate vs. silica) to give 1.33 g (6.2 mmol, 80%) of 4-(1H-pyrazol-3-yl)-2-trifluoromethyl-pyrimidine as an orange solid. ¹H-NMR (300 MHz, CDCl₃, δ): 9.59 (br s, 1H), 8.92 (d, J=5 Hz, 1H), 8.04 (d, J=5 Hz, 1H), 7.76 (d, J=3 Hz, 1H), 7.15 (d, J=2 Hz, 1H); m/z: 215 [M+H]⁺.

### (d) 4-(4-Bromo-1H-pyrazol-3-yl)-2-(trifluoromethyl)-pyrimidine

To an ice-cold, stirred solution of 1.33 g (6.2 mmol) of 4-(1H-pyrazol-3-yl)-2-trifluoromethyl-pyrimidine in 40 mL chloroform was added 0.63 mL (12.4 mmol) of bromine dropwise to give a red solution that developed an orange precipitate. This was warmed to room temperature and after 60 minutes the mixture was washed once with a saturated solution of NaHCO₃, then once with a 10% solution of sodium thiosulfate. The organic phase was dried (Na₂SO₄) and concentrated to give 1.31 g (4.5 mmol, 72%) of 4-(4-bromo-1H-pyrazol-3-yl)-2-trifluoromethyl-pyrimidine as a yellow solid. ¹H-NMR (300 MHz, CDCl₃, δ): 11.32 (br s, 1H), 9.01 (d, J=5 Hz, 1H), 8.41 (d, J=5 Hz, 1H), 7.67 (s, 1H); m/z: 294, 296 [M+H]⁺.

### (e) 4-(4-Bromo-1-(N,N-dimethylsulfamoyl)-pyrazol-3-yl)-2-(trifluoromethyl)-pyrimidine

To a stirred solution of 1.3 g (4.5 mmol) of 4-(4-bromo-1H-pyrazol-3-yl)-2-trifluoromethyl-pyrimidine in 20 mL dry DMF under nitrogen was added 0.27 g (6.6 mmol) of NaH (60% dispersion in oil) to give a green/brown mixture. This was stirred for 30 minutes, then 0.57 mL (5.3 mmol) of N,N-dimethylsulfamoyl chloride was added dropwise to give a dark brown mixture. This was stirred for 30 minutes, then quenched with water and diluted with ethyl acetate. The organic was washed twice with 1N NaOH, twice with a 5% citric acid solution, then brine, dried (Na₂SO₄) and concentrated. The residue was purified by flash chromatography (1:1 hexanes/ethyl acetate vs. silica) to give 1.40 g (3.5 mmol, 78%) of protected 4-bromo-3-(2-trifluoromethyl-pyrimidin-4-yl)-pyrazole-1-sulfonic acid dimethylamide as a pale yellow solid. ¹H-NMR (300 MHz, CDCl₃, δ): 9.00 (d, J=5 Hz, 1H), 8.14 (m, 2H), 3.05 (s, 6H); m/z: 401 [M+H]⁺.

### (f) 4-Benzo[1,3]dioxol-5-yl-3-(2-trifluoromethyl-pyrimidin-4-yl)-pyrazole-1-sulfonic acid dimethylamide

In a pressure tube was combined 200 mg (0.5 mmol) 4-bromo-3-(2-trifluoromethyl-pyrimidin-4-yl)-pyrazole-1-sulfonic acid dimethylamide, 125 mg (0.75 mmol) 3,4-(methylenedioxy)phenyl boronic acid (Aldrich Chemical Co., St. Louis, Mo.), and 35 mg (0.03 mmol, 6 mol %) of tetrakis-(triphenylphosphine)-palladium (0) (Strem Chemical, Newburyport, Mass.) which were suspended in 5 mL of dioxane with stirring. To this was added 1.5 mL 1M Na₂CO₃ solution, the tube flushed with argon and capped, and the resulting yellow mixture heated to 85° C. Upon reaching temperature, the mixture clarified to a yellow solution. This was stirred overnight, allowed to cool, and diluted with ethyl acetate. The organic was washed 3× with 1N NaOH, then brine, dried (NaₐSO₄), filtered and concentrated to form a pale yellow solid, 4-benzo[1,3]dioxol-5-yl-3-(2-trifluoromethyl-pyrimidin-4-yl)-pyrazole-1-sulfonic acid dimethylamide, which was used in the next step without further purification.

### (g) 4-(4-Benzo[1,3]dioxol-5-yl-1H-pyrazol-3-yl)-2-trifluoromethyl-pyrimidine

In a pressure tube was dissolved the residue of 4-benzo[1,3]dioxol-5-yl-3-(2-trifluoromethyl-pyrimidin-4-yl)-pyrazole-1-sulfonic acid dimethylamide in 4 mL (excess) of 0.5M NaOMe in MeOH and 1 mL THF and the tube was capped and heated to 85° C. with stirring overnight. The resulting yellow solution was cooled to ambient temp., neutralized with glacial AcOH, then subjected to reverse-phase preparatory HPLC (H₂O/acetonitrile, no buffer; 5% AcCN to 80% AcCN over 10 minutes) to give 20 mg (0.07 mmol, 25%) of 4-(4-benzo[1,3]dioxol-5-yl-1H-pyrazol-3-yl)-2-trifluoromethyl-pyrimidine as a yellow fluffy solid following lyophilization. LC-MS/ES+: M+1:335. ¹H-NMR (300 MHz, CDCl₃, δ): 11.50 (br s, 1H), 8.77 (d, J=6 Hz, 1H), 7.68 (s, 1H), 7.59 (d, J=6 Hz, 1H), 6.90 (m, 3H), 6.06 (s, 2H); m/z: 335 [M+H]⁺.

The compounds listed in the following Table were prepared in an analogous manner to those described in the Schemes and Examples above. The NMR and mass spectroscopy data of these compounds are included in the Table.

| | | | MS | |
|---|---|---|---|---|
| | | | (ES+) | |
| | | | m/z | |
| Example | Chemical Name | ¹H-NMR | (M + 1) | Method |
| Ex. 8 | 7-[3-(2-Trifluoromethyl- | 300 MHz, DMSO-d₆, δ: | 332 [M + H]⁺. | Scheme 4, |
| | pyrimidin-4-yl)-1H- | 13.92 (br s, 1H), 9.06 (d, J = 6 Hz, | | Example 7 |
| | pyrazol-4-yl]- | 1H), 8.67 (d, J = 7 Hz, 1H), | | |
| | [1,2,4]triazolo[1,5- | 8.48 (s, 1H), 8.40 (br s, 1H), | | |
| | a]pyridine | 8.23 (br s, 1H), 7.99 (m, 1H), | | |
| | | 7.30 (dd, J = 2 Hz, 7 Hz, 1H) | | |
| Ex. 9 | 6-[3-(2-Trifluoromethyl- | 300 MHz, DMSO-d₆, δ): | 342 [M + H]⁺. | Scheme 4, |
| | pyrimidin-4-yl)-1H- | 13.87 (br s, 1H), 9.03 (d, J = 5 Hz, | | Example 7 |
| | pyrazol-4-yl]-quinoline | 1H), 8.91 (d, J = 4 Hz, 1H), | | |
| | | 8.34 (d, J = 8 Hz, 1H), 8.26 (m, | | |
| | | 2H), 8.18 (s, 1H), 7.98 (d, J = 9 Hz, | | |
| | | 1H), 7.90 (d, J = 9 Hz, 1H), | | |
| | | 7.57 (q, J = 4 Hz, 1H) | | |

The TGFβ or activin inhibitory activity of compounds of formula (I) can be assessed by methods described in the following examples.

### EXAMPLE 10

### P Cell-Free Assay for Evaluating Inhibition of Autophosphorylation of TGFβ Type I Receptor

The serine-threonine kinase activity of TGFβ type I receptor was measured as the autophosphorylation activity of the cytoplasmic domain of the receptor containing an N-terminal poly histidine, TEV cleavage site-tag, e.g., His-TGFβRI. The His-tagged receptor cytoplasmic kinase domains were purified from infected insect cell cultures using the Gibco-BRL FastBac HTb baculovirus expression system.

To a 96-well Nickel FlashPlate (NEN Life Science, Perkin Elmer) was added 20 μL of 1.25 μCi ³³P-ATP/25 μM ATP in assay buffer (50 mM Hepes, 60 mM NaCl, 1 mM MgCl₂, 2 mM DTT, 5 mM MnCl₂, 2% glycerol, and 0.015% Brij® 35). 10 μL of test compounds of formula (I) prepared in 5% DMSO solution were added to the FlashPlate. The assay was then initiated with the addition of 20 μL of assay buffer containing 12.5 μmol of His-TGFβRI to each well. Plates were incubated for 30 minutes at room temperature and the reactions were then terminated by a single rinse with TBS. Radiation from each well of the plates was measured using TopCount (PerkinElmer Lifesciences, Inc., Boston Mass.). Total binding (no inhibition) was defined as counts measured in the presence of DMSO solution containing with no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGFβRI SDS-PAGE band was quantitated on a Storm Phosphoimager (Molecular Dynamics).

Compounds of formula (I) typically exhibited IC₅₀ values of less than 10 μM; some exhibited IC₅₀ values of less than 1.0 μM; and some even exhibited IC₅₀ values of less than 0.1 μM.

### EXAMPLE 11

### Cell-Free Assay for Evaluating Inhibition of Activin Type I Receptor Kinase Activity

Inhibition of the Activin type I receptor (Alk 4) kinase autophosphorylation activity by test compounds of formula (I) can be determined in a similar manner as described above in Example 10 except that a similarly His-tagged form of Alk 4 (His-Alk 4) can be used in place of the His-TGFβRI.

### EXAMPLE 12

### TGFβ Type I Receptor Ligand Displacement FlashPlate Assay

50 nM of tritiated 4-(3-pyridin-2-yl-1H-pyrazol-4-yl)-quinoline (custom-ordered from PerkinElmer Life Science, Inc., Boston, Mass.) in assay buffer (50 mM Hepes, 60 mM NaCl₂, 1 mM MgCl₂, 5 mM MnCl₂, 2 mM 1,4-dithiothreitol (DTT), 2% Brij® 35; pH 7.5) was premixed with a test compound of formula (I) in 1% DMSO solution in a v-bottom plate. Control wells containing either DMSO without test compound or control compound in DMSO were used. To initiate the assay, His-TGFβ Type I receptor in the same assay buffer (Hepes, NaCl₂, MgCl₂, MnCl₂, DTT, and 30% Brij® added fresh) was added to nickel coated FlashPlate (PE, NEN catalog number: SMP107), while the control wells contained only buffer (i.e., no His-TGFβ Type I receptor). The premixed solution of tritiated 4-(3-pyridin-2-yl-1H-pyrazol-4-yl)-quinoline and test compound of formula (I) was then added to the wells. The wells were aspirated after an hour at room temperature and radioactivity in wells (emitted from the tritiated compound) was measured using TopCount (PerkinElmer Lifesciences, Inc., Boston Mass.).

Compounds of formula (I) typically exhibited Kᵢ values of less than 10 μM; some exhibited Kᵢ values of less than 1.0 μM; and some even exhibited Kᵢ values of less than 0.1 μM.

### EXAMPLE 13

### Assay for Evaluating Cellular Inhibition of TGFβ Signaling and Cytotoxicity

Biological activity of compounds of formula (I) were determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/mL), streptomycin (100 μg/mL), L-glutamine (2 mM), sodium pyruvate (1 mM), and non essential amino acids (1×). The transfected cells were then plated at a concentration of 2.5×10⁴ cells/well in 96 well plates and starved for 3-6 hours in media with 0.5% FBS at 37° C. in a 5% CO₂ incubator. The cells were then stimulated with ligand either 2.5 ng/mL TGFβ in the starvation media containing 1% DMSO and the presence or absence of test compounds of formula (I) and incubated as described above for 24 hours. The media was washed out in the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of formula (I) for inhibiting TGFβ-induced PAI-Luciferase reporter activity in HepG2 cells. Compounds of formula (I) typically exhibited IC₅₀ values of less 10 μM.

Cytotoxicity was determined using the same cell culture conditions as described above. Specifically, cell viability was determined after overnight incubation with the CytoLite cell viability kit (Packard, cat. no. 6016901). Compounds of formula (I) typically exhibited LD₂₅ values greater than 10 μM.

### EXAMPLE 14

### Assay for Evaluating Cellular Inhibition of TGFβ Signaling

The cellular inhibition of activin signaling activity by test compounds of formula (I) are determined in a similar manner as described above in Example 13 except that 100 ng/mL of activin can be added to serum starved cells in place of the 2.5 ng/mL TGFβ.

### EXAMPLE 15

### Assay for TGFβ-Induced Collagen Expression

### Preparation of Immortalized Collagen Promotor-Green Fluorescent Protein Cells

Fibroblasts are derived from the skin of adult transgenic mice expressing Green Fluorescent Protein (GFP) under the control of the collagen 1A1 promoter (see Krempen, K. et al., Gene Exp. 8: 151-163 (1999)). Cells are immortalised with a temperature sensitive large T antigen that is active at 33° C. Cells are expanded at 33° C. then transferred to 37° C. so that the large T becomes inactive (see Xu, S. et al., Exp. Cell Res. 220: 407-414 (1995)). Over the course of about 4 days and one split, the cells cease proliferating. Cells are then frozen in aliquots sufficient for a single 96 well plate.

Assay of TGFβ-induced Collagen-GFP Expression

Cells are thawed, plated in complete DMEM (contains nonessential amino acids, 1 mM sodium pyruvate and 2 mM L-glutamine) with 10% fetal calf serum and incubated overnight at 37° C., 5% CO₂. The following day, the cells are trypsinized and transferred into 96 well format with 30,000 cells per well in 50 μL complete DMEM containing 2% fetal calf serum, but without phenol red. The cells are incubated at 37° C. for 3 to 4 hours to allow them to adhere to the plate, solutions containing test compounds of formula (I) are then added to triplicate wells with no TGFβ, as well as triplicate wells with 1 ng/mL TGFβ. DMSO was also added to all of the wells at a final concentration of 0.1%. GFP fluorescence emission at 530 nm following excitation at 485 nm was measured at 48 hours after the addition of solution containing test compounds on a CytoFluor microplate reader (PerSeptive Biosystems). The data are then expressed as the ratio of TGFβ-induced to non-induced for each test sample.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of formula (I): 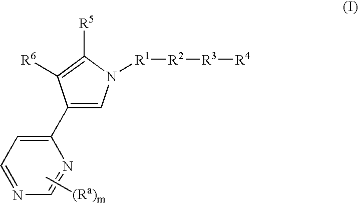
or an N-oxide or a pharmaceutically acceptable salt thereof;
wherein
each R^{a}, independently, is alkyl, alkenyl, alkynyl, alkoxy, acyl, halo, hydroxy, —NH₂, —NH(unsubstituted alkyl), —N(unsubstituted alkyl)₂, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, cycloalkylcarbonyl, heterocycloalkyl, heterocycloalkyloxy, heterocycloalkylsulfanyl, heterocycloalkylcarbonyl, aryloxy, arylsulfonyl, aroyl, heteroaryl, heteroaryloxy, heteroarylsulfonyl, or heteroaroyl;
R¹ is a bond, alkylene, alkenylene, alkynylene, or —(CH₂)ᵣ₁—O—(CH₂)ᵣ₂—, where each of r1 and r2, independently, is 2 or 3;
R² is cycloalkylene, heterocycloalkylene, cycloalkenylene, heterocycloalkenylene, arylene, heteroarylene, or a bond;
R³
is —C(O)—, —C(O)—O—, —O—C(O)—, —S(O)ₚ—O—, —O—S(O)ₚ—, —C(O)—N(R^{b})—, —N(R^{b})—C(O)—, —O—C(O)—N(R^{b})—, —N(R^{b})—C(O)—O—, —C(O)—N(R^{b})—O—, —O—N(R^{b})—C(O)—, —O—S(O)ₚ—N(R^{b})—, —N(R^{b})—S(O)ₚ—O—, —S(O)ₚ—N(R^{b})—O—, —O— N(R^{b})—S(O)ₚ—, —N(R^{b})—C(O)—N(R^{c})—, —N(R^{b})—S(O)ₚ—N(R^{c})—, —C(O)—N(R^{b})—S(O)ₚ—, —S(O)ₚ—N(R^{b})—C(O)—, —C(O)—N(R^{b})—S(O)ₚ—N(R^{c})—, —C(O)—O—S(O)ₚ—N(R^{b})—, —N(R^{b})—S(O)ₚ—N(R^{c})—C(O)— —N(R^{b})—S(O)ₚ—C(O)—, —S(O)ₚ—N(R^{b})—, —N(R^{b})—S(O)ₚ—, —N(R^{b})—, —S(O)ₚ—, —O—, —S—, —(C(R^{b})(R^{c}))_{q}—, or a bond; wherein each of R^{b} and R^{c} is independently hydrogen, hydroxy, alkyl, aryl, aralkyl, heterocycloalkyl, heteroaryl, or heteroaralkyl; p is 1 or 2; and q is 1-4;
R⁴ is hydrogen, alkyl, alkenyl, alkynyl, cycloalkyl, (cycloalkyl)alkyl, heterocycloalkyl, (heterocycloalkyl)alkyl, cycloalkenyl, (cycloalkenyl)alkyl, heterocycloalkenyl, (heterocycloalkenyl)alkyl, aryl, aralkyl, heteroaryl, or heteroaralkyl;
R⁵ is hydrogen, unsubstituted alkyl, halo-substituted alkyl, alkoxy, alkylsulfinyl, amino, alkenyl, alkynyl, cycloalkoxy, cycloalkylsulfinyl, heterocycloalkoxy, heterocycloalkylsulfinyl, aryloxy, arylsulfinyl, heteroaryloxy, or heteroarylsulfinyl;
R⁶ is a 5- to 6-membered monocyclic heterocyclyl or a 8- to 11-membered bicyclic heteroaryl; each being optionally substituted with alkyl, alkenyl, alkynyl, alkoxy, acyl, halo, hydroxy, amino, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, heterocycloalkyl, heterocycloalkyloxy, heterocycloalkylsulfanyl, cycloalkylcarbonyl, heterocycloalkylcarbonyl, aryl, aryloxy, arylsulfonyl, aroyl, heteroaryl, heteroaryloxy, heteroarylsulfonyl, or heteroaroyl; and
m is 0-3; provided that when m≧2, two adjacent R^{a} groups can join together to form a 4- to 8-membered optionally substituted cyclic moiety.

2. The compound of claim 1, wherein R⁶ is a 5- to 6-membered heterocyclyl containing 1-3 hetero ring atoms selected from the group consisting of —O—, —S—, —N═, and —NR^{d}—, where R^{d} is hydrogen, alkyl, cycloalkyl, cycloalkylalkyl, aryl, aralkyl, heterocycloalkyl, heterocycloalkylalkyl, heteroaryl, or heteroaralkyl; said heterocyclyl being optionally substituted with one to two R^{f}; where R^{f} is alkyl, alkenyl, alkynyl, alkoxy, acyl, halo, hydroxy, amino, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, cycloalkylcarbonyl, heterocycloalkyl, heterocycloalkyloxy, heterocycloalkylsulfanyl, heterocycloalkylcarbonyl, aryl, aryloxy, arylsulfonyl, aroyl, heteroaryl, heteroaryloxy, heteroarylsulfonyl, or heteroaroyl.

3. The compound of claim 2, wherein R⁶ is a 5- to 6-membered heterocyclyl containing 1-3 hetero ring atoms selected from the group consisting of —O—, —S—, —N═, and —NR^{d}— where R^{d} is hydrogen or alkyl.

4. The compound of claim 3, wherein R⁶ is a 6-membered heteroaryl containing 1 or 2 hetero ring atoms wherein each hetero ring atom is —N═ or —NR^{d}—.

5. The compound of claim 4, wherein R⁶ is or 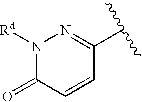
or

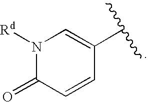

6. The compound of claim 1, wherein R⁶ is a fused ring heteroaryl selected from the group consisting of: 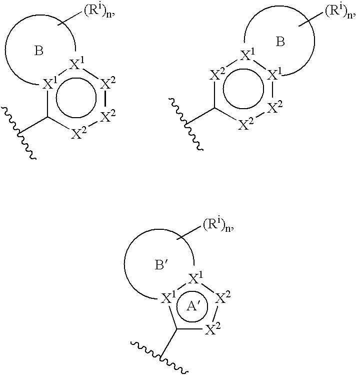
and 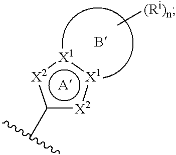
where ring A is an aromatic ring containing 0-4 hetero ring atoms, and ring B is a 5- to 7-membered aromatic or nonaromatic ring containing 0-4 hetero ring atoms; provided that at least one of ring A and ring B contains one or more hetero ring atoms; ring A′ is an aromatic ring containing 0-4 hetero ring atoms, and ring B′ is a 5- to 7-membered saturated or unsaturated ring containing 0-4 hetero ring atoms; provided that at least one of ring A′ and ring B′ contains one or more hetero ring atoms; each hetero ring atom is —O—, —S—, —N═, or —NR^{g}—; each X¹ is independently N or C; each X² is independently —O—, —S—, —N═, —NR^{g}—, or —CHR^{h}—; where R^{g} is hydrogen, alkyl, cycloalkyl, cycloalkylalkyl, aryl, aralkyl, heterocycloalkyl, heterocycloalkylalkyl, heteroaryl, or heteroaralkyl; each of R^{h} and Rⁱ is independently hydrogen, alkyl, alkenyl, alkynyl, alkoxy, acyl, halo, hydroxy, amino, nitro, oxo, thioxo, cyano, guanidino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfonyl, cycloalkylcarbonyl, heterocycloalkyl, heterocycloalkyloxy, heterocycloalkylsulfanyl, heterocycloalkylcarbonyl, aryl, aryloxy, arylsulfonyl, aroyl, heteroaryl, heteroaryloxy, heteroarylsulfonyl, or heteroaroyl; and n is 0-2.

7. The compound of claim 6, wherein R⁶ is 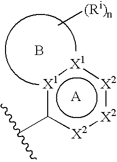
or

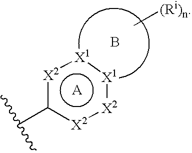

8. The compound of claim 7, wherein ring B is a 5- to 6-membered aromatic or nonaromatic ring.

9. The compound of claim 7, wherein R⁶ contains at least two hetero ring atoms.

10. The compound of claim 7, wherein R⁶ contains at least three hetero ring atoms.

11. The compound of claim 9 or 10, wherein the para-position of ring A is occupied by or substituted with one of said hetero ring atoms or the para-position of ring A is substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—CO—N(R^{j})₂ where each R^{j} is independently hydrogen, alkyl, cycloalkyl, cycloalkylalkyl, aryl, aralkyl, heterocycloalkyl, heterocycloalkylalkyl, heteroaryl, or heteroaralkyl.

12. The compound of claim 8, wherein R⁶ is 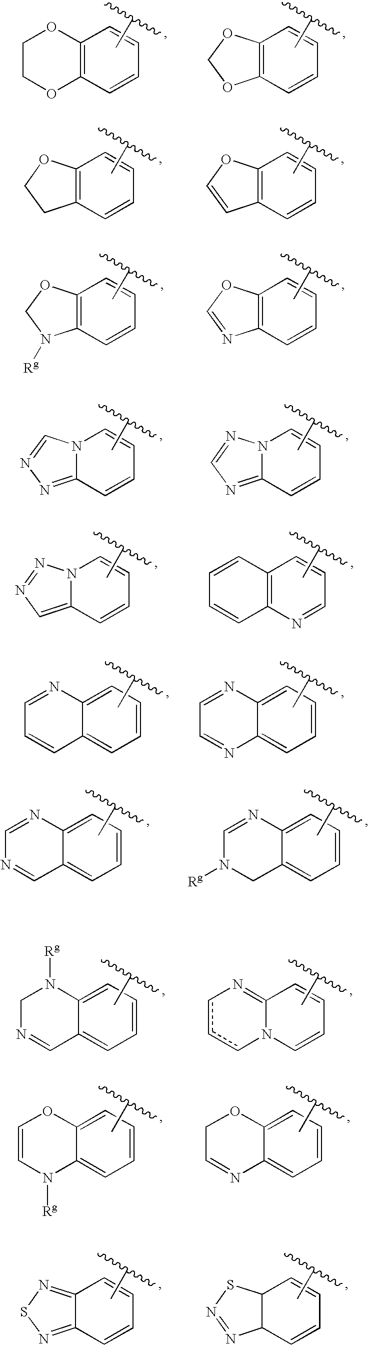 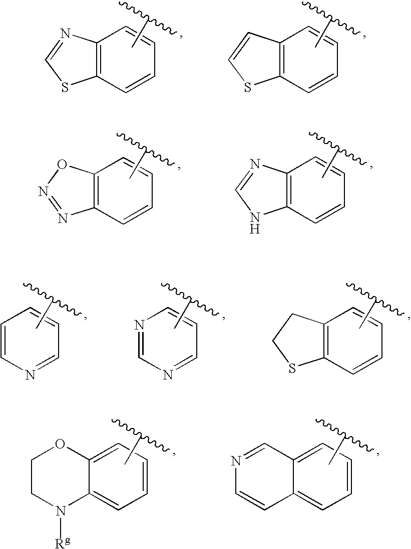
or 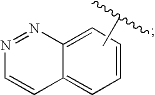
each of which being optionally substituted with alkyl, alkoxy, halo, oxo, thioxo, amino, alkylsulfinyl, cyano, carboxy, aryl, or heteroaryl and R^{g} being hydrogen, alkyl, cycloalkyl, cycloalkylalkyl, aryl, aralkyl, heterocycloalkyl, heterocycloalkylalkyl, heteroaryl, or heteroaralkyl.

13. The compound of claim 12, wherein R⁶ is 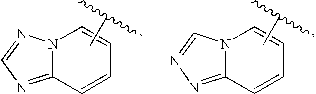
or

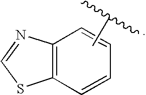

14. The compound of claim 13, wherein R⁶ is 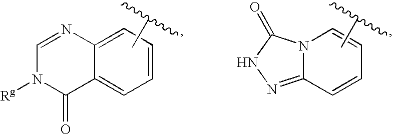
or

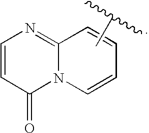

15. The compound of claim 13, wherein R⁶ is 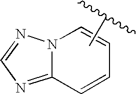
or

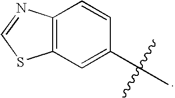

16. The compound of claim 6, wherein R⁶ is 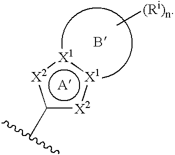
or

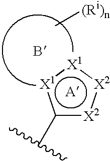

17. The compound of claim 16, wherein ring B′ is a 5- to 6-membered aromatic or nonaromatic ring.

18. The compound of claim 16, wherein R⁶ contains at least two hetero ring atoms.

19. The compound of claim 16, wherein R⁶ contains at least three hetero ring atoms.

20. The compound of claim 17, wherein R⁶ is 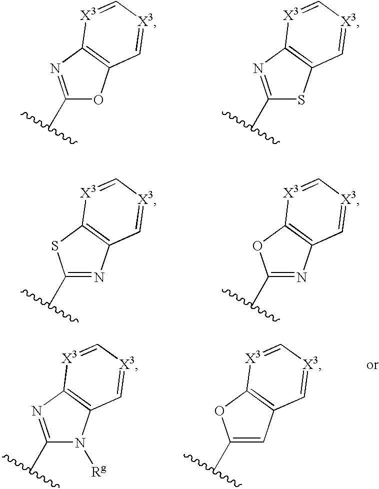
or 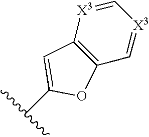
wherein X³ is independently N or C; and each R⁶ is optionally substituted with alkyl, alkoxy, halo, oxo, thioxo, amino, alkylsulfinyl, cyano, carboxy, aryl, or heteroaryl.

21. The compound of claim 1, wherein R² is a bond, alkylene, or —(CH₂)₂—O—(CH₂)₂—.

22. The compound of claim 1, wherein R² is cycloalkylene, heterocycloalkylene, arylene, heteroarylene, or a bond.

23. The compound of claim 1, wherein R³
is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, —N(R^{b})—C(O)—O, —C(O)—N(R^{b})—O—, —N(R^{b})—C(O)—N(R^{c})—, —C(O)—N(R^{b})—S(O)ₚ—N(R^{c})—, —C(O)—O—S(O)ₚ—N(R^{b})—, or a bond.

24. The compound of claim 1, wherein R⁴ is hydrogen, alkyl, cycloalkyl, heterocycloalkyl, aryl, or heteroaryl.

25. The compound of claim 1, wherein R¹ is a bond or alkylene; R² is a bond; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S(O)ₚ—N(R^{b})—, —N(R^{b})—, or a bond; and R⁴ is hydrogen, alkyl, cycloalkyl, heterocycloalkyl, aryl, or heteroaryl.

26. The compound of claim 1, wherein R¹ is a bond or alkylene; R² is a bond; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S(O)ₚ—N(R^{b})—, —N(R^{b})—, or a bond; and R⁴ is hydrogen, alkyl, cycloalkyl, or heterocycloalkyl.

27. The compound of claim 1, wherein R¹ is —(CH₂)₂—O—(CH₂)₂—; R² is piperidinylene, piperazinylene, pyrrolidinylene, tetrahydrofuranylene, tetrahydropyranylene, tetrahydrothiopyranylene, tetrahydrothiopyranylene-1-oxide, tetrahydrothiopyranylene-1-dioxide, cyclohexylene, cyclopentylene, bicyclo[2.2.1]heptanylene, bicyclo[2.2.2]octanylene, bicyclo[3.2.1]octanylene, 2-oxa-bicyclo[2.2.2]octanylene, 2-aza-bicyclo[2.2.2]octanylene, 3-aza-bicyclo[3.2.1]octanylene, cubanylene, or 1-aza-bicyclo[2.2.2]octanylene; R³ is a bond; and R⁴ is hydrogen, alkyl, cycloalkyl, heterocycloalkyl, aryl, or heteroaryl.

28. The compound of claim 1, wherein R¹ is a bond; R² is piperidinylene, piperazinylene, pyrrolidinylene, tetrahydrofuranylene, tetrahydropyranylene, tetrahydrothiopyranylene, tetrahydrothiopyranylene-1-oxide, tetrahydrothiopyranylene-1-dioxide, cyclohexylene, cyclopentylene, bicyclo[2.2.1]heptanylene, bicyclo[2.2.2]octanylene, bicyclo[3.2.1]octanylene, 2-oxa-bicyclo[2.2.2]octanylene, 2-aza-bicyclo[2.2.2]octanylene, 3-aza-bicyclo[3.2.1]octanylene, cubanylene, or 1-aza-bicyclo[2.2.2]octanylene; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, or a bond; and R⁴ is hydrogen, alkyl, cycloalkyl, heterocycloalkyl, aryl, or heteroaryl.

29. The compound of claim 1, wherein each of R¹, R², and R³ is a bond; and R⁴ is hydrogen.

30. The compound of claim 1, wherein each of R¹ and R³ is a bond; R² is cycloalkylene, heterocycloalkylene, or a bond; and R⁴ is hydrogen, cycloalkyl, or heterocycloalkyl.

31. The compound of claim 1, wherein R⁵ is hydrogen, unsubstituted alkyl, or halo-substituted alkyl.

32. The compound of claim 1, wherein R⁵ is hydrogen.

33. The compound of claim 1, wherein m is 0, 1, or 2.

34. The compound of claim 1, wherein m is 1 or 2 and at least one R^{a} is substituted at the 2-pyrimidinyl position.

35. The compound of claim 1, wherein each R^{a} is independently alkyl, alkoxy, alkylsulfinyl, halo, amino, aminocarbonyl, alkoxycarbonyl, cycloalkyl, or heterocycloalkyl.

36. The compound of claim 1, wherein each R^{a} is independently unsubstituted alkyl, halo-substituted alkyl, C₃₋₆ cycloalkyl, or 3- to 6-membered heterocycloalkyl.

37. The compound of claim 1, wherein R⁶ is 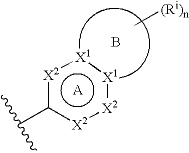
in which ring B is a 5- to 6-membered aromatic or nonaromatic ring; R⁵ is hydrogen, unsubstituted alkyl, or halo-substituted alkyl; R⁴ is hydrogen, alkyl, heterocycloalkyl, aryl, or heteroaryl; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, or a bond; R² is a bond; R¹ is a bond or alkylene; and R^{a} is alkyl, cycloalkyl, or heterocycloalkyl; provided that if m is not 0, at least one R^{a} is substituted at the position in between the two nitrogen ring atoms.

38. The compound of claim 37, wherein the para-position of ring A is occupied by or substituted with a hetero ring atom or the para-position of ring A is substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—CO—N(R^{j})₂ where each R^{j} is independently hydrogen, alkyl, cycloalkyl, cycloalkylalkyl, aryl, aralkyl, heterocycloalkyl, heterocycloalkylalkyl, heteroaryl, or heteroaralkyl.

39. The compound of claim 37, wherein R⁶ is 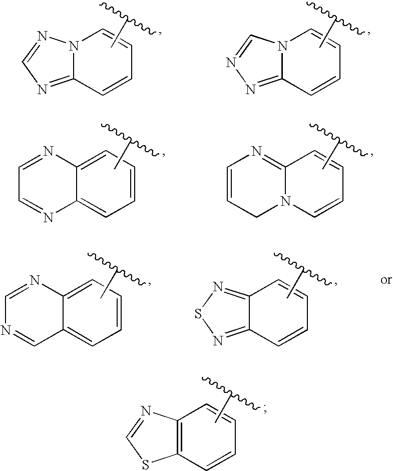
or 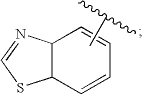
each of which being optionally substituted with alkyl, alkoxy, halo, hydroxy, oxo, amino, alkylsulfinyl, cyano, carboxy, aryl, or heteroaryl.

40. The compound of claim 39, wherein R⁶ is 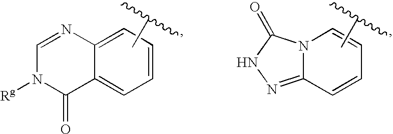
or 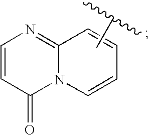
each of which being optionally substituted with alkyl, alkoxy, halo, hydroxy, oxo, amino, alkylsulfinyl, cyano, carboxy, aryl, or heteroaryl.

41. The compound of claim 37, wherein R⁴ is hydrogen or alkyl; R³ is —N(R^{b})—C(O)—, —N(R^{b})—S(O)ₚ—, —C(O)—N(R^{b})—, —S(O)ₚ—N(R^{b})—, —N(R^{b})—, or a bond; R² is cycloalkylene or a bond; R¹ is a bond, alkylene, or —(CH₂)₂—O—(CH₂)₂—.

42. The compound of claim 41, wherein R⁴-R³-R²-R¹- is hydrogen.

43. The compound of claim 40, wherein R⁵ is hydrogen, unsubstituted methyl, or trifluoromethyl.

44. The compound of claim 43, wherein R⁵ is hydrogen.

45. The compound of claim 1, said compound being selected from the group consisting of: 4-(4-benzo[1,3]dioxol-5-yl-1H-pyrazol-3-yl)-2-methyl-pyrimidine, 6-[3-(2-methyl-pyrimidin-4-yl)-1H-pyrazol-4-yl]-[1,2,4]triazolo[1,5-a]pyridine, 6-[3-(2-trifluoromethyl-pyrimidin-4-yl)-1H-pyrazol-4-yl]-[1,2,4]triazolo[1,5-a]pyridine, 6-[3-(2-methyl-pyrimidin-4-yl)-1H-pyrazol-4-yl]-quinoxaline, 6-[3-(2-trifluoromethyl-pyrimidin-4-yl)-1H-pyrazol-4-yl]-quinoxaline, 6-[3-(2-cyclopropyl-pyrimidin-4-yl)-1H-pyrazol-4-yl]-quinoxaline, 4-(4-benzo[1,3]dioxol-5-yl-1H-pyrazol-3-yl)-2-trifluoromethyl-pyrimidine, 7-[3-(2-trifluoromethyl-pyrimidin-4-yl)-1H-pyrazol-4-yl]-[1,2,4]triazolo[1,5-a]pyridine, and 6-[3-(2-Trifluoromethyl-pyrimidin-4-yl)-1H-pyrazol-4-yl]-quinoline.

46. The compound of claim 1, said compound being selected from the group consisting of: 6-[3-(2-methyl-pyrimidin-4-yl)-1H-pyrazol-4-yl]-quinoxaline, 6-[3-(2-methyl-pyrimidin-4-yl)-1H-pyrazol-4-yl]-[1,2,4]triazolo[1,5-a]pyridine, and 4-(4-benzo[1,3]dioxol-5-yl-1H-pyrazol-3-yl)-2-methyl-pyrimidine.

47. A pharmaceutical composition comprising a compound of claim 1 and a pharmaceutically acceptable carrier.

48. A pharmaceutical composition comprising a compound of claim 45 and a pharmaceutically acceptable carrier.

49. A method of inhibiting the TGFβ signaling pathway in a subject, comprising administering to said subject with an effective amount of a compound of claim 1.

50. A method of inhibiting the TGFβ signaling pathway in a subject, comprising administering to said subject with an effective amount of a compound of claim 45.

51. A method of inhibiting the TGFβ type I receptor in a cell, comprising contacting said cell with an effective amount of a compound of claim 1.

52. A method of inhibiting the TGFβ type I receptor in a cell, comprising contacting said cell with an effective amount of a compound of claim 45.

53. A method of reducing the accumulation of excess extracellular matrix induced by TGFβ in a subject, comprising administering to said subject an effective amount of a compound of claim 1.

54. A method of reducing the accumulation of excess extracellular matrix induced by TGFβ in a subject, comprising administering to said subject an effective amount of a compound of claim 45.

55. A method of treating or preventing fibrotic condition in a subject, comprising administering to said subject an effective amount of a compound of claim 1.

56. A method of treating or preventing fibrotic condition in a subject, comprising administering to said subject an effective amount of a compound of claim 45.

57. The method of claim 55 or 56, wherein the fibrotic condition is induced by radiation.

58. The method of claim 55 or 56, wherein the fibrotic condition is selected from the group consisting of scleroderma, lupus nephritis, connective tissue disease, wound healing, surgical scarring, spinal cord injury, CNS scarring, acute lung injury, idiopathic pulmonary fibrosis, radiation-induced pulmonary fibrosis, chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, fatty liver disease, primary sclerosing cholangitis, restenosis, cardiac fibrosis, ophthalmic scarring, fibrosclerosis, a fibrotic cancer, a fibroid, fibroma, a fibroadenoma, a fibrosarcoma, transplant arteriopathy, and keloid.

59. A method of inhibiting growth or metastasis of tumor cells or cancer in a subject, comprising administering to said subject an effective amount of a compound of claim 1.

60. A method of inhibiting growth or metastasis of tumor cells or cancer in a subject, comprising administering to said subject an effective amount of a compound of claim 45.

61. A method of treating a disease or disorder mediated by an overexpression of TGFβ, comprising administering to a subject in need of such treatment an effective amount of a compound of claim 1.

62. A method of treating a disease or disorder mediated by an overexpression of TGFβ, the method comprising administering to a subject in need of such treatment an effective amount of a compound of claim 45.

63. The method of claim 61 or 62, wherein the disease or disorder is selected from the group consisting of demyelination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, and head and neck.
